(19) United States
(12) Patent Application Publication
(10) Pub. No.: US 2026/0182847 A1
(43) Pub. Date: Jul. 2, 2026

(54) Multi-Sensor Differential Measurement for Blood Pressure Determination

(71) Applicant: iRhythm Technologies, Inc., San Francisco, CA (US)

(72) Inventors: Monica C. Lin, San Francisco, CA (US); Dylan Reinsdorf, San Francisco, CA (US); Avirath Dodabele, New York, NY (US)

(73) Assignee: iRhythm Technologies, Inc., San Francisco, CA (US)

(21) Appl. No.: 19/436,883

(22) Filed: Dec. 30, 2025

Related U.S. Application Data
(60) Provisional application No. 63/740,297, filed on Dec. 30, 2024.

(51) Int. Cl. A61B 5/021; A61B 5/00; A61B 5/024; A61B 5/318

(52) U.S. Cl. CPC A61B 5/02125; A61B 5/02416; A61B 5/318; A61B 5/7203; A61B 2562/04; A61B 2562/06

(57) ABSTRACT

Techniques for multi-sensor differential measurement for blood pressure determination are described. In one or more implementations, sensor data is received from a plurality of sensors positioned at different locations on a body. A time delay of a physiological event at two or more locations on the body is determined based on the sensor data. At least one differential measurement between the two or more locations is calculated based on the time delay. A blood pressure measurement is determined based on the at least one differential measurement.

### RELATED APPLICATIONS

This application claims priority to U.S. Provisional Application No. 63/740,297, filed Dec. 30, 2024, and titled “Multi-Sensor Differential Measurement for Blood Pressure Determination,” which is hereby incorporated by reference in its entirety.

### BACKGROUND

Blood pressure monitoring devices traditionally rely on oscillometric cuffed systems that provide intermittent measurements through inflatable cuffs placed around a limb. However, cuffed systems are susceptible to measurement errors from factors such as incorrect cuff sizing, suboptimal cuff shape design, and motion artifacts during measurement cycles. Moreover, cuffed systems obtain blood pressure data at discrete measurement intervals rather than through continuous monitoring, which constrains the temporal resolution of blood pressure data collection. Additionally, the form factor and operational characteristics make cuffed systems unsuitable for extended wear periods or integration into compact wearable platforms. Alternative approaches for cuffless blood pressure measurement have focused on pulse arrival time measurements between electrocardiogram signals and photoplethysmography signals, but these methods may suffer from poor accuracy if not related to another physiological parameter such as impedance cardiography. However, including multiple sensor types may increase circuit complexity.

### BRIEF DESCRIPTION OF THE DRAWINGS

FIG. 1 is a block diagram of a non-limiting example of an environment that is operable to employ techniques for multi-sensor differential measurement for blood pressure determination as described herein.

FIG. 2 depicts a non-limiting example of a monitoring device.

FIG. 3 illustrates a non-limiting example showing different sensor configurations (e.g., placement arrangements) on a person for multi-sensor differential measurement techniques to estimate blood pressure.

FIG. 4 illustrates a method for estimating blood pressure using differential measurements.

FIG. 5 includes an example of the first experimental setup for comparing PPG signal quality and PTT values at different sensor locations.

FIG. 6 depicts an example that includes ECG and PPG recordings from the monitoring device positioned at the chest location and the additional monitoring device positioned at the top position along the carotid artery at the neck during a first data collection period.

FIG. 7 depicts an example that compares pulse transit time as calculated using PPG peaks, denoted PTTp, for the first data collection period.

FIG. 8 depicts an example that includes ECG and PPG recordings from the monitoring device positioned at the chest location and the additional monitoring device positioned at the middle position along the carotid artery at the neck during a second data collection period.

FIG. 9 depicts an example that compares pulse transit time as calculated using PPG peaks, denoted PTTp, for the second data collection period.

FIG. 10 depicts an example that includes ECG and PPG recordings from the monitoring device positioned at the chest location and the additional monitoring device positioned at the bottom position along the carotid artery at the neck during a third data collection period.

FIG. 11 depicts an example that compares pulse transit time as calculated using PPG peaks, denoted PTTp, for the third data collection period.

FIG. 12 illustrates an example of expected measurement behaviors during a Valsalva maneuver.

FIG. 13 illustrates examples comparing pulse transit time metrics calculated from data measured at multiple points along the carotid artery at the neck and blood pressure data during a Valsalva maneuver.

FIG. 14 depicts examples comparing pulse transit time metrics calculated from data measured at multiple points along the carotid artery at the neck and blood pressure data during resistance band exercises.

### DETAILED DESCRIPTION

Blood pressure may be used as an indicator of cardiovascular health, but traditional measurement techniques face challenges. Conventional cuff-based devices inflate around a limb during scheduled cycles to obtain intermittent readings and can be uncomfortable for patients to wear, especially during sleep or daily activities. Moreover, the intermittent readings may not capture variations throughout daily activities and may miss short lived changes related to stress, exertion, and/or sleep transitions, for example.

Non-invasive, continuous blood pressure monitoring techniques have been explored using various physiological signals and sensors placed on the body. Accurately estimating blood pressure without a cuff, however, remains challenging due to individual variations in physiology, sensor placement issues, and complex relationships between measurable parameters and actual blood pressure values. By way of example, cuffless blood pressure measurement approaches have explored pulse arrival time (PAT) measurements that calculate a time delay between an electrocardiogram (ECG) R-peak and a photoplethysmogram (PPG) feature to estimate blood pressure. This PAT time delay has been shown to be inversely correlated with blood pressure, as both are related to arterial wall elasticity. However, these conventional pulse arrival time-based measurements may be affected by pre-ejection period (PEP) variations and other cardiac timing factors that can introduce measurement errors. By way of example, because the PEP can vary (e.g., due to contractility, sympathetic activation, stress, exercise, etc.), PAT may not accurately correlate with blood pressure unless the PEP is accounted for.

To account for PEP-related variability, traditional cuffless blood pressure measurement approaches may use additional sensors, such as impedance cardiography (ICG) sensors, to estimate the PEP and/or other phases of the blood flow cycle to refine blood pressure estimates. However, this mixed-modality sensing (e.g., having at least three different sensor modalities in this example) introduces heterogeneous analog front-end characteristics, added power demands in compact wearables, and complicates circuit design.

Accordingly, techniques for multi-sensor differential measurement for blood pressure determination are described herein to overcome the limitations of conventional techniques. In an example, sensor data is received from a plurality of sensors positioned at different locations on a body. As used herein, “different locations” may refer to sensors positioned at different regions of the body (e.g., the chest and the neck) or sensors positioned at a same region of the body but spaced apart (e.g., in a clustered arrangement for sampling at different tissue depths or in a geometric pattern for triangulation). A time delay of a physiological event, such as an arrival of a pulse wave, is determined at two or more locations on the body based on the sensor data. By way of example, the time delay of the physiological event may be determined by detecting characteristic features (e.g., a peak or foot/trough) in PPG waveforms captured by the sensors in comparison to a reference (e.g., a cardiac timing reference, such as the R-peak of an ECG). As used herein, the “arrival” may refer to any feature of the pulse wave, such as the arrival of the PPG peak, the arrival of the PPG foot, the arrival of any inflection point along the PPG waveform between the PPG peak and the PPG foot, and so forth. At least one differential measurement is calculated between the two or more locations based on the time delay at the two or more locations. The at least one differential measurement may be referred to herein as a differential pulse transit time (PTT) between two corresponding locations (e.g., one proximal to the heart and one distal to the heart). A blood pressure measurement is determined based on the at least one differential measurement.

In some examples, the sensors include at least two PPG sensors, such as a first PPG sensor at a proximal position relative to a heart and a second PPG sensor at a distal position relative to the heart. Additional sensor types such as ECG, ICG, or accelerometry sensors may also be incorporated. Sensor placement locations can include the upper chest, the lower chest, the neck region, the forehead, an upper arm, a forearm, a wrist, a finger, a leg, or a foot. In some configurations, three or more PPG sensors may be positioned to triangulate the blood pressure measurement, and the PPG sensors may be positioned in a variety of configurations to achieve a variety of advantages further described herein.

To ensure accuracy, the sensor data may undergo processing steps (e.g., via one or more algorithms) that include noise filtering, removal of low-quality data, and application of statistical analyses over time series data. The techniques described herein allow for continuous, non-invasive blood pressure monitoring that can capture variations throughout daily activities and sleep. By eliminating a reliance on an inflatable cuff, the techniques described herein provide a comfortable experience for users while improving measurement frequency and consistency. The multi-sensor differential technique further provides redundancy and supports robust determinations compared to conventional single-point measurement systems and reduces circuit complexity compared to systems that include additional sensor types rather than a plurality of PPG sensors.

As used herein, the term “continuous” used in connection with measurements, such as PPG measurements, ECG measurements, and the like, may refer to an ability of a device to produce measurements substantially continuously, such that the device may be configured to produce the output measurements at intervals of time (e.g., per hour, per 30 minute interval, per 5 minute interval, per 30 second interval, per second, per half second, and so forth), responsive to an event (e.g., an electrical signal reaching an inflection point such as a peak or a valley), and so forth. The functionality of the device to produce the measurements and/or to record any of a variety of signals may vary without departing from the spirit or scope of the described techniques.

In some aspects, the techniques described herein relate to a method for estimating blood pressure, including: receiving sensor data from a plurality of sensors positioned at different locations on a body; determining a time delay of a physiological event at two or more locations on the body based on the sensor data; calculating at least one differential measurement between the two or more locations based on the time delay; and determining a blood pressure measurement based on the at least one differential measurement.

In some aspects, the techniques described herein relate to a method, wherein the plurality of sensors includes at least two photoplethysmography (PPG) sensors positioned at the different locations on the body.

In some aspects, the techniques described herein relate to a method, wherein the plurality of sensors further includes at least one of an electrocardiography (ECG) sensor, an impedance cardiography (ICG) sensor, or an accelerometry sensor.

In some aspects, the techniques described herein relate to a method, wherein the at least two PPG sensors include a first PPG sensor at a proximal position relative to a heart and a second PPG sensor at a distal position relative to the heart.

In some aspects, the techniques described herein relate to a method, wherein determining the time delay of the physiological event at the two or more locations on the body based on the sensor data includes: detecting a characteristic feature in first sensor data obtained at a first of the two or more locations on the body; detecting the characteristic feature in second sensor data obtained at a second of the two or more locations on the body; and determining the time delay of the physiological event based on respective timings of the detected characteristic feature in the first sensor data and the second sensor data.

In some aspects, the techniques described herein relate to a method, wherein the physiological event includes an arrival of a pulse wave at a corresponding location of the two or more locations on the body.

In some aspects, the techniques described herein relate to a method, wherein the different locations on the body include one or more of an upper chest, a lower chest, a neck region, a forehead, an upper arm, a forearm, a wrist, a finger, a leg, or a foot.

In some aspects, the techniques described herein relate to a method, wherein the plurality of sensors includes at least three PPG sensors.

In some aspects, the techniques described herein relate to a method, wherein the at least three PPG sensors are positioned in a configuration to triangulate the blood pressure.

In some aspects, the techniques described herein relate to a method, wherein the plurality of sensors includes at least two PPG sensors positioned to obtain light reflectance measurements received from different depths of a same area on the body.

In some aspects, the techniques described herein relate to a method, further including processing the sensor data prior to determining the time delay by: filtering the sensor data to remove noise; and removing portions of the sensor data that are below a quality threshold.

In some aspects, the techniques described herein relate to a method, wherein determining the blood pressure measurement based on the at least one differential measurement includes applying one or more statistical analyses to the at least one differential measurement over a plurality of heartbeats.

In some aspects, the techniques described herein relate to a system for estimating blood pressure, including: a plurality of sensors configured for placement at different locations on a body, including: a first photoplethysmography (PPG) sensor configured to be positioned at a first location on the body; and a second PPG sensor configured to be positioned at a second location on the body; and at least one processor configured to: determine a differential measurement based on a time delay of a physiological event that is determined based on a characteristic feature detected by the first PPG sensor and a corresponding characteristic feature detected by the second PPG sensor; and determine a blood pressure measurement based on the differential measurement.

In some aspects, the techniques described herein relate to a system, wherein the plurality of sensors further includes at least one electrocardiography (ECG) sensor.

In some aspects, the techniques described herein relate to a system, wherein the plurality of sensors further includes a third PPG sensor configured to be positioned at a third location on the body that is selected relative to the first location and the second location to enable triangulation of the blood pressure measurement.

In some aspects, the techniques described herein relate to a system, wherein the different locations include one or more of an upper chest, a lower chest, a neck region, a forehead, an upper arm, a forearm, a wrist, a finger, a leg, or a foot.

In some aspects, the techniques described herein relate to a system, wherein to determine the differential measurement based on the time delay of the physiological event, the at least one processor is further configured to: detect the characteristic feature in a first PPG waveform obtained by the first PPG sensor; detect the corresponding characteristic feature in a second PPG waveform obtained by the second PPG sensor; and calculate the differential measurement based on respective timings of the characteristic feature in the first PPG waveform and the corresponding characteristic feature in the second PPG waveform.

In some aspects, the techniques described herein relate to a method for estimating blood pressure, including: receiving a first photoplethysmography (PPG) waveform from a first PPG sensor and a second PPG waveform from a second PPG sensor that is spaced apart from the first PPG sensor on a body; determining a time delay of a physiological event based on respective timings of a characteristic feature in the first PPG waveform and a corresponding characteristic feature in the second PPG waveform; calculating a differential measurement based on the time delay; and determining a blood pressure measurement based on the differential measurement.

In some aspects, the techniques described herein relate to a method, wherein determining the time delay of the physiological event based on the respective timings of the characteristic feature in the first PPG waveform and the corresponding characteristic feature in the second PPG waveform includes: determining, based on the first PPG waveform, a first pulse transit time (PTT) corresponding to a first time delay for a pulse wave to reach the first PPG sensor; and determining, based on the second PPG waveform, a second PTT corresponding to a second time delay for the pulse wave to reach the second PPG sensor, and wherein the differential measurement is a difference between the first PTT and the second PTT.

In some aspects, the techniques described herein relate to a method, wherein determining the time delay of the physiological event based on the respective timings of the characteristic feature in the first PPG waveform and the corresponding characteristic feature in the second PPG waveform includes calculating a time difference between the characteristic feature in the first PPG waveform and the corresponding characteristic feature in the second PPG waveform.

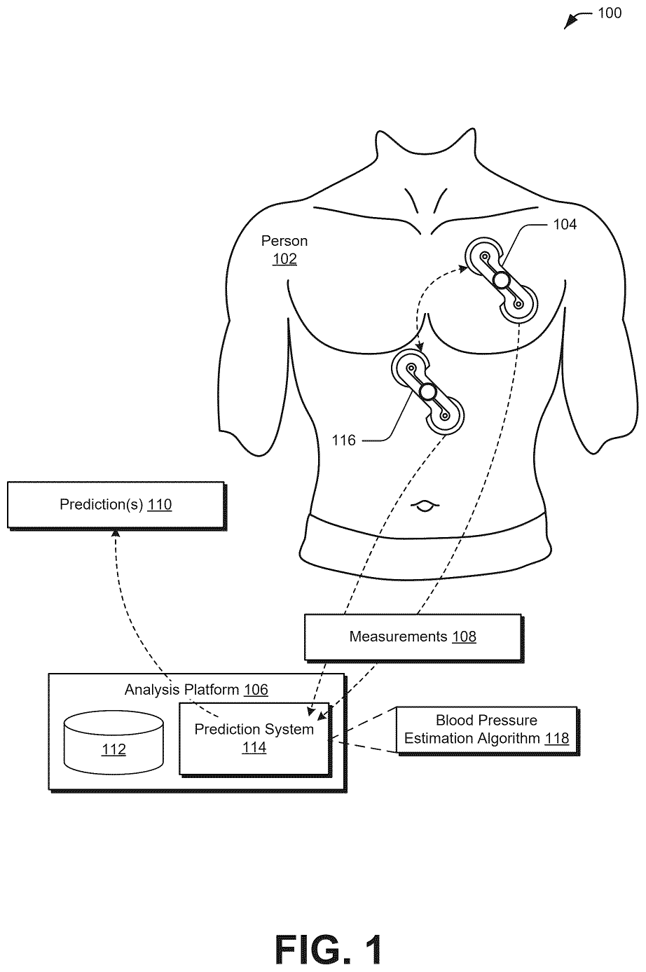

FIG. 1 is a block diagram of a non-limiting example 100 of an environment that is operable to employ techniques for multi-sensor differential measurement for blood pressure determination as described herein. The illustrated example 100 includes a person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102. By way of example, the monitoring device 104 may be utilized to monitor one or more of ECG, electroencephalography (EEG), electromyography (EMG), respiratory inductance plethysmography (RIP), pulse oximetry, accelerometry, impedance cardiography (ICG), or the like as measurements 108. The monitoring device 104 of the non-limiting example 100 includes a PPG sensor for non-invasive monitoring of various physiological parameters. By way of example, the PPG sensor may be utilized to monitor one or more of pulse rate, heart rate variability, blood oxygen saturation, respiration, blood volume, blood perfusion, and blood pressure. The PPG sensor may comprise one or more light sources, such as light-emitting diodes (LEDs) and/or laser diodes, and one or more photodetectors. The one or more light sources may emit light at one or more wavelengths to monitor the various physiological parameters. By way of example, the one or more light sources may emit light in the red to infrared spectrum, which penetrates the skin and underlying tissues more efficiently than light having shorter wavelengths (e.g., light within the ultraviolet to orange regions of the spectrum). In at least one variation, however, the one or more light sources of the PPG sensor emit light of a shorter wavelength (e.g., green light) in addition to or as an alternative to the longer wavelength light. In one or more implementations, the PPG sensor is configured to emit light at and detect multiple different wavelengths of light to capture different physiological parameters.

As the heart pumps blood through the body, the volume of blood in the microvascular bed of the tissue fluctuates. The PPG sensor detects these volume changes by measuring the amount of light absorbed or reflected by the blood vessels. The photodetector captures the reflected or transmitted light, which varies with each heartbeat, allowing the device to measure parameters such as pulse rate, blood oxygen saturation, and pulse wave characteristics.

In one or more implementations, the monitoring device 104 may combine PPG sensing with other modalities, such as electrocardiography and/or impedance cardiography, to provide a more comprehensive picture of the physiological state of the person 102. This multi-modal approach may enhance the ability of the monitoring device 104 to detect and monitor various health conditions, including sleep disorders, arrhythmias, hypertension, or changes in cardiovascular function.

In some scenarios, for instance, the monitoring device 104 may be provided to record electrical activity of the heart of the person 102 over an observation period, e.g., lasting some number of seconds or minutes, lasting multiple days, and so on. By way of example, the person 102 may have a magnitude of his or her heart's electrical potential monitored over time to produce one or more electrocardiograms, which may be used to predict any of a variety of events. Alternatively, or in addition, the monitoring device 104 may be used to output the measurements 108 (e.g., a time sequence of measurements such as a time sequence of electric potential measurements), which may indicate an observation or be used to generate an assessment, diagnosis, or prediction of one or more events.

In connection with the monitoring device, instructions may be provided to the person 102 that instruct the person 102 how to operate the monitoring device 104 and/or how to behave (e.g., sleep, perform activity) while wearing monitoring device 104. In one or more implementations, the instructions may be provided as part of a kit, e.g., as written instructions. Alternatively, or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of an observation period has lapsed (e.g., two days) while wearing the monitoring device 104 and/or based on patterns in the aspects of the person 102 being measured.

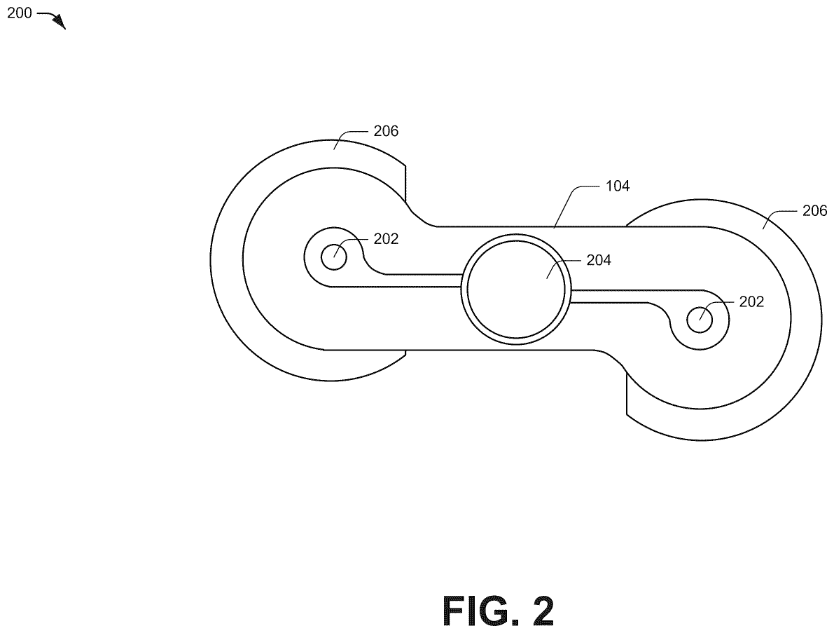

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, an ICG sensor, and a PPG sensor (e.g., to measure and record oxygen saturation (SpO2) and/or produce a photoplethysmogram of the person 102), to name just a few. Certainly, the monitoring device 104 may be configured with any of a variety of types of sensors without departing from the described techniques.

Although the monitoring device 104 may be configured in a similar manner to monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively, or additionally, the monitoring device 104 may have a similar form factor as for clinical settings, but may have different functionality, such as functionality that prevents a wearer from viewing the measurements 108.

In one or more implementations, the monitoring device 104 may be configured to offload the measurements 108 and/or other data during the course of the observation period. By way of example, the monitoring device 104 may offload the measurements 108 by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of sleep apnea.

To the extent that the monitoring device 104 may be configured to store the measurements 108 for an entirety of an observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from the storage of the device.

Accordingly, the monitoring device 104 may be configured with one or more ports to enable wired transmission of the measurements to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternatively or additionally be configured to offload the measurements 108 over one or more wireless connections.

Once the monitoring device 104 produces the measurements 108, the measurements are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from local storage of the device to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured to generate the one or more predictions 110. For example, the measurements 108 (and/or other measurements such as accelerometer data and oxygen saturation (SpO2) measurements) may be processed by a smartphone associated with a user (e.g., the person 102 or another person associated with the person 102), a smartphone or other dedicated device associated with the monitoring device 104, and/or one or more server computers at a data center or other location that can be utilized by an entity associated with the monitoring device 104, to name just a few.

In one or more implementations, the monitoring device 104 is configured to transmit the measurements 108 to an external device over a wired connection with the external device, e.g., via USB-C or some other physical, communicative coupling. Here, a connector may be plugged into the monitoring device 104 or the monitoring device 104 may be inserted into an apparatus having a receptacle that interfaces with corresponding contacts of the device. The measurements 108 may then be obtained from storage of the monitoring device 104 via this wired connection, e.g., transferred over the wired connection to the external device. Such a connection may be used in scenarios where the monitoring device 104 is mailed by the person 102 after the observation period, such as to a health care provider, telemedicine service, provider of the monitoring device 104, or medical testing laboratory.

Alternatively, or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with external devices using one or more wireless communication protocols or techniques. By way of example, the monitoring device 104 may communicate with external devices using one or more of Bluetooth® (e.g., Bluetooth® Low Energy links), near-field communication (NFC), Long Term Evolution (LTE™) standards such as 5G, and so forth. The monitoring device 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smart watch, chest strap, or the like. As noted above, examples of such additional measurements include but are not limited to accelerometer data and/or oxygen saturation measurements.

In one or more implementations, the analysis platform 106 may be implemented in whole or in part at the monitoring device 104. Alternatively, or in addition, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform 106 includes a storage device 112 and a prediction system 114. In accordance with the described techniques, the storage device 112 is configured to maintain the measurements 108 and/or other measurements or information processed by the prediction system 114 to generate the one or more predictions 110. The storage device 112 may represent one or more databases and/or other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population.

In the illustrated example 100, the prediction system 114 represents functionality to process the measurements 108 to generate the one or more prediction(s) 110. In at least one implementation, the one or more predictions 110 may include an assessment or diagnosis related to a health condition or disease state. Alternatively, or in addition, the prediction system 114 may output one or more time sequences indicating an observation or prediction of one or more events over time. It is also to be appreciated that, in variations, the prediction system 114 may output different combinations of multiple predictions.

In at least one implementation, the prediction system 114 uses machine learning to generate at least a portion of the one or more predictions 110. By way of example and not limitation, the prediction system 114 may include one or more neural networks trained based on the historical measurements and the historical outcome data of a user population. The prediction system 114 may include one or multiple machine learning models (e.g., an ensemble of models). Alternatively, or additionally, the prediction system 114 may include logic (a machine learning model and/or other types of logic) to pre-process the measurements 108, such as to extract various cardiovascular and/or other features from the sequences of measurements. In the illustrated example 100, for instance, the one or more predictions 110 correspond to the output of the prediction system 114.

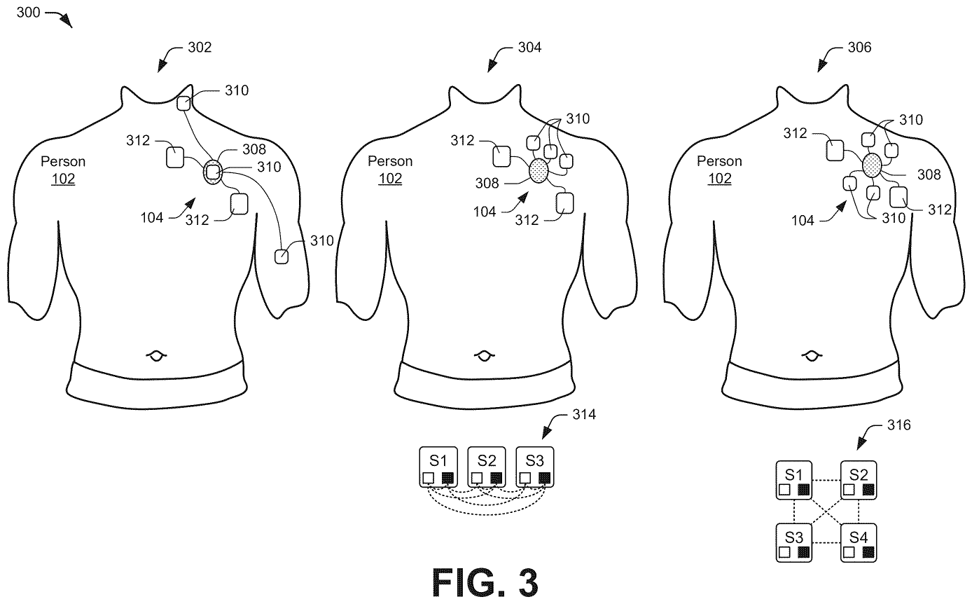

The example 100 is further depicted as including an additional monitoring device 116. Although different reference numerals are used, the additional monitoring device 116 may share one or more properties and/or functionality and/or communication capabilities with the monitoring device 104. In some implementations, the monitoring device 104 may include a first sensor, while the additional monitoring device 116 may include a second sensor. In at least one variation, the monitoring device 104 includes both the first sensor and the second sensor. By way of example, the second sensor may be distally positioned from a housing of the monitoring device 104 but connected to the housing (e.g., by a wire or flexible circuit trace), such as will be further described with respect to FIG. 3. It will be appreciated that the first sensor and second sensor described herein may be capable of one or more of ECG, EEG, EMG, RIP, ICG, PPG, accelerometry, or the like as the measurements 108. Analysis of data from both sensors may provide a blood pressure measurement. For example, the first sensor may be positioned at a proximal location on the body relative to the heart, while the second sensor may be positioned at a distal location relative to the heart. The time delay between corresponding physiological events detected by these sensors may be used to calculate a differential measurement, which in turn may be used to determine blood pressure.

In some implementations, the monitoring device 104 provides a first PPG signal from a location nearer (e.g., proximal) to the heart of the person 102, and the additional monitoring device 116 provides a second PPG signal from a location farther (e.g., distal) from the heart. Time differences between corresponding pulse features in these PPG signals may be computed as differential pulse transit time values that serve as inputs to determine the blood pressure. This arrangement may also support use of more than two sensing locations to provide redundancy and to allow the prediction system 114 to select signal pairs of higher quality. By way of example, the monitoring device 104 may include more than one PPG sensor, and the additional monitoring device 116 may include at least one additional PPG sensor. In one or more implementations, the prediction system 114 may evaluate signal quality for all available PPG sensors to select the pair or pairs that provide the highest quality differential pulse transit time values.

Although only one additional monitoring device 116 is shown in the illustrated example, it should be understood that multiple additional monitoring devices may be included in various implementations. These additional devices may share one or more properties, functionality, and/or communication capabilities as the monitoring device 104 and/or the additional monitoring device 116. These additional devices may be positioned at different locations on the body to capture a range of physiological measurements. Multiple locations may enable triangulation across different paths, sampling at different tissue depths or angles, and/or an automatic selection of sensor pairs with higher signal quality to improve robustness during movement and daily activities. In some examples, subsets of the processing described for the analysis platform 106 may execute on the monitoring device 104 and/or on the additional monitoring device 116.

In the illustrated example 100, the analysis platform 106 further includes a blood pressure estimation algorithm 118. The blood pressure estimation algorithm 118 represents functionality of the prediction system 114 to analyze the measurements 108 obtained at different sensing locations (e.g., from the monitoring device 104 and/or the additional monitoring device 116) to determine blood pressure values and trends based on differential measurements between the different sensing locations. Continuing the above example where the monitoring device 104 provides the first PPG signal and the additional monitoring device 116 provides the second PPG signal, the blood pressure estimation algorithm 118 may determine time differences between corresponding pulse features in the first PPG signal and the second PPG signal, which may provide differential pulse transit time values that are further used to estimate blood pressure values, indices, and trends. In one or more implementations, these values may be output as the one or more predictions 110.

In some cases, the blood pressure estimation algorithm 118 may assess signal quality, segment a heartbeat, and/or average values across beats. Alternatively, or in addition, the blood pressure estimation algorithm 118 may utilize ECG data to assist with segmentation and quality gating. A mapping from differential pulse transit time to blood pressure may be implemented using one or more models, which may include machine learning models trained on reference data and/or parametric models that relate vascular properties to timing.

In one or more implementations, the prediction system 114 performs a real-time analysis of the measurements 108 obtained by the monitoring device 104 and/or the additional monitoring device 116 to determine the blood pressure. As used herein, “real-time” may refer to processing that occurs without intentional delay (e.g., the measurements 108 are analyzed substantially as acquired) and produces updated outputs with a latency that is short relative to cardiac dynamics. In some cases, this corresponds to updating the differential pulse transit time and corresponding blood pressure values within milliseconds, seconds, and/or within about one to three beats. Alternatively, or in addition, the prediction system 114 may perform a near real-time analysis, where the measurements 108 are processed with a delay of seconds or minutes. A near real-time analysis may reduce power consumption of the monitoring device 104 and/or the additional monitoring device 116 and may be suitable for blood pressure applications that use blood pressure measurements on a scale of hours rather than seconds. In at least one implementation, the blood pressure estimation algorithm 118 may receive streaming data of the measurements 108, align timestamps across sensing locations, segment heartbeats, extract pulse features, and compute time differences between corresponding features as each beat is detected. The blood pressure estimation algorithm 118 may apply sliding windows to stabilize estimates while preserving responsiveness, update confidence metrics, and/or gate values using quality assessments. Alternatively, or additionally, the blood pressure estimation algorithm 118 may switch among sensor pairs based on real-time quality and motion determinations. Alternatively, or in addition, the blood pressure estimation algorithm 118 may use a moving average, a median, a weighted average (e.g., that more heavily weights more recent values), or another statistical technique applied to the differential pulse transit time measurements over a configurable number of heartbeats to mitigate the effects of physiological (e.g., respiratory) artifacts. Moreover, the blood pressure estimation algorithm 118 may exclude any heartbeat for which the PPG signal quality is less than a quality threshold, which may be configurable (e.g., by a user) and/or learned via a machine learning model.

In some cases, a portion of these computations execute on-device (e.g., on the monitoring device 104 and/or the additional monitoring device 116) to reduce latency and power, and a portion executes at the analysis platform 106 when connectivity is available, which may support continuous operation during daily wear. Accordingly, in at least one implementation, an entirety or portions of the blood pressure estimation algorithm 118 may be executed at the monitoring device 104, the analysis platform 106, and/or the additional monitoring device 116.

In this way, the illustrated example 100 enables differential blood pressure determination based on multi-location photoplethysmogram timing. The analysis platform 106, through the blood pressure estimation algorithm 118, may coordinate acquisition of the measurements 108 from the monitoring device 104 and/or the additional monitoring device 116, align data across sensing locations, compute the differential pulse transit time (e.g., in real-time, at least in one implementation), and generate the one or more predictions 110 with quality gating and adaptive sensor-pair selection to improve cuffless continuous blood pressure monitoring robustness. This approach may provide flexibility in device configurations, as the blood pressure estimation algorithm 118 may be implemented at the monitoring device 104, at the additional monitoring device 116, at the analysis platform 106, and/or at a separate device associated with one or more of these devices. By leveraging multiple PPG sensors positioned at different body locations, the illustrated example 100 may support continuous blood pressure monitoring with improved stability, enable values and trends to be determined over extended wear, and simplify the analog front end through same-modality sensing (e.g., two PPG sensors) in a manner that reduces power consumption and calibration overhead of the monitoring device 104 while simplifying circuitry.

FIG. 2 depicts a non-limiting example 200 of a monitoring device. The illustrated example 200 depicts the monitoring device 104. As described above, in various examples, the additional monitoring device 116 may include one or more properties and/or features of the monitoring device 104.

In accordance with the described techniques, the monitoring device 104 includes one or more sensors 202, examples of which include but are not limited to one or more pairs of electrodes, an accelerometer, a PPG sensor, and sweat sensors, to name just a few. The monitoring device 104 may also include a transmitter 204. In this example 200, the monitoring device 104 further includes one or more adhesive portions 206. In operation, the monitoring device 104 is configured to be applied to the skin via the one or more adhesive portions 206, such that, for example, the one or more sensors 202 are positioned to detect and record the electrical activity of the heart of the person 102, e.g., to produce an electrocardiogram (ECG or EKG). In at least one implementation, the monitoring device 104 may be removed by peeling the one or more adhesive portions 206 off of the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on the communications with one or more sensors 202 that are indicative of some aspect of the person 102, such as the electrical activity of the heart of the person 102. In one or more implementations, the processor further generates one or more communicable packages of data that include one or more of the measurements 108 and/or other measurements, such as ECG and PPG measurements. Alternatively, or additionally, the processor produces and/or causes storage of other data, which may be used for monitoring various physiological states of the person 102.

In implementations where the monitoring device 104 is configured for wireless transmission, the transmitter 204 may transmit the measurements wirelessly as a stream of data to a computing device (e.g., the analysis platform 106). In one or more implementations, for instance, the monitoring device 104 is configured to transfer (e.g., transmit and/or receive) information (e.g., ECG and/or PPG measurements) via a Bluetooth® Low Energy (BLE) connection. Alternatively, or additionally, the monitoring device 104 may buffer the measurements (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

### Multi-Sensor Differential Measurement Techniques for Blood Pressure Determination

FIG. 3 illustrates a non-limiting example 300 showing different sensor configurations (e.g., placement arrangements) on a person 102 for multi-sensor differential measurement techniques to estimate blood pressure. The example 300 depicts three example configurations of the monitoring device 104 on the torso of the person 102, indicated as a first example configuration 302, a second example configuration 304, and a third example configuration 306. It is to be appreciated that although the example 300 includes the monitoring device 104 having multiple sensors positioned at different locations on the person 102, in at least one variation, a portion of the sensors may be included in a separate device (e.g., the additional monitoring device 116).

In each example configuration, the monitoring device 104 comprises a device housing 308, which may house associated electronics, and a plurality of sensors. In the example 300, the monitoring device 104 includes PPG sensors 310 and electrodes 312. That is, the monitoring device 104 includes more than one PPG sensor 310 and more than one electrode 312 in the example 300. Each PPG sensor 310 may be configured to gather PPG data, and the electrodes 312 may be configured to gather electrical data, such as ECG data, EMG data, and/or ICG data. The first example configuration 302, the second example configuration 304, and the third example configuration 306 demonstrate alternative spatial arrangements for capturing physiological signals usable for differential pulse transit time and blood pressure calculations.

In the first example configuration 302, the monitoring device 104 is positioned on the upper chest region of the person 102, and the sensors are arranged in a distributed pattern. A distributed pattern may allow simultaneous measurement of electrical heart activity (e.g., via the electrodes 312) and blood flow (e.g., via the PPG sensors 310) at multiple points on the body of the person 102, which may broaden coverage and reduce blind spots in data collection. The distributed pattern of the first example configuration 302 may be selected for performance of, for example, whole-body monitoring.

The first example configuration 302 includes three PPG sensors 310 positioned at distinct locations on the body of the person 102. For instance, a first PPG sensor 310 is positioned on or near the neck of the person 102 (e.g., over the carotid artery), a second PPG sensor 310 is overlapping with or within the device housing 308 on the center left of the upper chest of the person 102, and a third PPG sensor 310 is positioned on the left arm of the person 102. It will be appreciated that any number of PPG sensors 310 may be used, and the PPG sensors 310 may be positioned at different locations on a body of the person 102 without departing from the spirit or scope of the described techniques. The arrangement of the PPG sensors 310 in the first example configuration 302 may provide additional timing information by “tracking” the flow of blood over the length of a vessel network. As another example, the long distance between the PPG sensors 310 in the first example configuration 302 may provide an improved or more sensitive signal, such as by providing a larger differential measurement (e.g., a greater amplitude of signal).

The first example configuration 302 further includes the electrodes 312 positioned on the chest of the person 102. In one or more implementations, the electrodes 312 may be arranged to establish a measurement vector across a cardiac axis. The electrodes 312 are configured to contact a skin surface to acquire electrical cardiac signals that may be used, e.g., for heartbeat segmentation, timing reference, and quality assessment in connection with differential pulse transit time. The electrodes 312 may be implemented as dry electrodes, hydrogel electrodes, fabric electrodes, or other electrode constructions and may be included on an adhesive portion or on a strap. The locations of the electrodes 312 may be varied to accommodate different body shapes while maintaining an adequate vector. Using different locations for the electrodes 312 may also accommodate different vectors, which may give different physiological information about the person 102. Additional electrodes 312 may be used to form additional or alternative lead configurations, at least in some implementations. Accordingly, the electrodes 312 may be arranged in various ways without departing from the spirit or scope of the described techniques.

In the second example configuration 304, the monitoring device 104 is similarly positioned on the chest of the person 102 and includes the electrodes 312 arranged similarly to the first example configuration 302 described above. The monitoring device 104 of the second example configuration 304 includes the PPG sensors 310 in a clustered arrangement for sampling blood flow at different tissue depths. In reflectance-based photoplethysmography, light emitted from an emitter (e.g., a light source, such as an LED) travels through tissue and is scattered back for detection by a detector (e.g., by an optical sensor, such as a photodetector or photodiode). The depth that the light penetrates into the tissue before detection depends at least in part on the spacing between the emitter and the detector. When the emitter and the detector are closer together, the light follows a relatively shallow path through superficial tissue layers before detection. When the emitter and the detector are spaced farther apart, the light travels deeper into the tissue before detection, thus sampling blood flow in deeper vascular beds.

The second example configuration 304 includes a depth information diagram 314 illustrating how light emitted by one PPG sensor 310 may be detected by other PPG sensors 310 in the cluster via cross-sensor pairings. In this illustrative example, each PPG sensor 310 includes an emitter, represented by a white or open-filled square, and a detector, represented by a black-filled square. It is to be appreciated that a given PPG sensor 310 may include more than one emitter and/or more than one detector in variations. The PPG sensors 310 are labeled as S1, S2, and S3, and dashed lines indicate different PPG readings (e.g., measurements, such as values or waveforms) that may be obtained through different emitter-detector combinations having different emitter-to-detector spacings. By way of example, light emitted by the S1 emitter may result in three PPG readings of different depths: a first PPG reading based on light detected at the S1 detector, a second PPG reading based on light detected at the S2 detector (which is spaced farther from the S1 emitter than the S1 detector), and a third PPG reading based on light detected at the S3 detector (which is spaced farther from the S1 emitter than the S1 detector and the S2 detector). Alternatively, or in addition, light emitted by the S2 emitter or the S3 emitter may be similarly detected by the S1 detector, the S2 detector, and the S3 detector. In one or more implementations, the emitters of the PPG sensors 310 may emit light in a selective, alternating, or sequential manner, such that light detected by a given detector can be attributed to a specific emitter. By way of example, the S1 emitter may emit light during a first time interval while the S2 emitter and the S3 emitter are inactive, allowing the S1 detector, the S2 detector, and the S3 detector to each capture readings attributable to the S1 emitter during the first time interval. The S2 emitter may emit light during a second time interval while the S1 emitter and the S3 emitter are inactive, and so forth. In at least one variation, one or more of the PPG sensors 310 may not include an emitter and rely on light emitted by an emitter of another PPG sensor 310 in the cluster. In at least one other variation, one emitter may be used during an entirety of a data collection period while the other emitters remain inactive. By way of example, the S1 emitter may be used while the S2 emitter and the S3 emitter remain inactive.

A PPG reading, for instance, may be characterized by the emitter-detector pairing used to obtain the PPG reading. As used herein, a PPG reading “from” a given PPG sensor may refer to a reading captured by a detector of the given PPG sensor, regardless of which emitter provided the light source. By way of example, a first PPG reading may be obtained using the S1 emitter and the S1 detector, a second PPG reading may be obtained using the S2 emitter and the S1 detector, a third PPG reading may be obtained using the S3 emitter and the S1 detector, and so forth. Accordingly, different PPG readings may refer to measurements made by the same PPG sensor with illumination provided by different light sources, measurements made by different PPG sensors with illumination provided by the same light source, or combinations thereof.

Accordingly, the PPG sensors 310 in the second example configuration 304 are arranged to capture additional information about blood flow at different tissue depths under the same area of skin, which may provide more comprehensive data for blood pressure determination. The depth information may also support the capture of volumetric or layered data that may be useful for three-dimensional (3D) mapping of the physiological signals.

In some implementations, the arrangement of sensors for detection at different depths can be combined with the distributed arrangement shown in the first example configuration 302. As a non-limiting example, the monitoring device 104 can be arranged in the second example configuration 304, and the additional monitoring device 116 can be positioned at a different location on the body of the person 102, with the PPG sensors 310 arranged in a distributed pattern. As described above, any number of the additional monitoring devices 116 can be implemented in combination with the monitoring device 104.

In the third example configuration 306, the monitoring device 104 is similarly positioned on the chest region of the person 102 and includes the electrodes 312 arranged similarly to the first example configuration 302 described above. The monitoring device 104 of the third example configuration 306 includes the PPG sensors 310 arranged in a triangulation pattern. Triangulation patterns use geometric relationships (e.g., angles and distances) between sensors to calculate more precise positions and orientations and may be used to improve localization accuracy, reduce error from single-sensor drift, and enable higher precision mapping of physiological signals. By way of example, PPG sensors 310 placed in a triangle or square may enable more accurate triangulation of the blood pressure value by sampling the same area across different angles, providing overlapping information for use by the blood pressure estimation algorithm 118. The third example configuration 306 may, therefore, enable multi-point measurements and may allow for a more accurate estimation of blood pressure values.

The third example configuration 306 includes a triangulation diagram 316 illustrating how the PPG sensors 310 may be arranged in a geometric pattern to triangulate the blood pressure measurement. In this illustrative example, four PPG sensors 310 are positioned at vertices of a square arrangement and labeled as S1, S2, S3, and S4, with each PPG sensor 310 including at least one emitter and at least one detector. Similar to the second example configuration 304, light emitted by an emitter of one PPG sensor 310 may be detected by a detector of at least one other PPG sensor 310 in the arrangement, providing PPG measurements across different spatial paths. For illustrative clarity, rather than showing different specific emitter-detector configurations (e.g., as in the depth information diagram 314 of the second example configuration 304), dashed lines indicate cross-sensor detection. By way of example, light emitted by the S1 emitter may be detected by the S1 detector, the S2 detector, the S3 detector, and the S4 detector. These cross-sensor measurements may provide overlapping information from different angles across the triangulation diagram 316, which may improve accuracy in the blood pressure estimation. In one or more implementations, the emitters may emit light in a selective, alternating, or sequential manner to enable attribution of detected light to a specific emitter, such as described above with respect to the second example configuration 304.

It will be appreciated that the multi-sensor geometric arrangement of the third example configuration 306 can be implemented alone or in combination with the distributed arrangement of the first example configuration 302 and/or the depth detection arrangement of the second example configuration 304. Moreover, although the third example configuration 306 includes four PPG sensors 310 arranged in a square, other geometries and sensor numbers are possible. By way of example, the triangulation may be performed using three PPG sensors 310 arranged in a triangle.

In each of the three configurations described above, the PPG sensors 310 include at least two PPG sensors that form a differential pair so that a pulse transit time (PTT) between two measurement locations may be calculated for use in blood pressure determination. The two measurement locations include a large enough physical distance between them to capture time-delayed information about blood flow from distal and proximal points along a blood vessel (e.g., an artery, a vein, capillaries, or another vessel network). Moreover, the use of more than two PPG sensors 310 may provide redundancy in case of poor sensor contact. By way of example, if one sensor experiences degraded contact with a skin surface due to factors such as sweat or hair, an additional sensor(s) may allow continued data collection and selection of a higher-quality pair. In one or more implementations, the monitoring device 104 may obtain the measurements 108 between all combinations of the PPG sensors 310, and the blood pressure estimation algorithm 118 may determine which pair or pairs have the highest quality signal. The blood pressure estimation algorithm 118 may use the pair having the highest quality signal as the differential pair for measuring differential transit time.

Example locations for placement of the PPG sensors 310 include one or more of an upper chest, a lower chest, a neck region, a forehead, an upper arm, a forearm, a wrist, a finger, a leg, or a foot, which may support flexibility in device design and capture of physiological signals from different parts of the body.

In one or more implementations, the monitoring device 104 further includes an impedance cardiography (ICG) sensor and/or an accelerometry sensor in addition to the PPG sensors 310 and the electrodes 312. These sensor types may provide complementary data to enhance blood pressure determination. For example, PPG sensing from the PPG sensors 310 may be used together with ECG data obtained by the electrodes 312 to calculate a pulse-arrival time (PAT), which is an interval between ventricular depolarization (e.g., an R-wave of an ECG waveform) and a peripheral arrival of the pulse. Alternatively, or in addition, the ICG sensor may measure thoracic impedance changes due to blood volume changes in the heart and great vessels, providing measurements such as stroke volume and cardiac output. ICG may be used to determine a pre-ejection period (PEP), which is a time between ventricular depolarization and the beginning of mechanical ejection into the aorta. A PAT of a peripheral site may also be represented as a sum of a PEP measurement with a PTT measurement recorded for the peripheral site. Combining PEP information with PAT and/or PTT information may improve vascular compliance modeling and may improve blood pressure estimates. By way of example, PAT measurements may provide complementary information to PTT for estimating blood pressure. ICG data may offer insights into cardiac output and stroke volume, which may correlate with vascular compliance and changes in blood pressure. Alternatively, or in addition, accelerometers may be used with any of the sensors described to detect motion, posture, and/or vibration and may be used to compensate for motion artifacts underlying PPG and ECG signals.

In this way, the analysis platform 106 may develop a more comprehensive view of a cardiovascular state of the person 102. This multi-modal approach may support robust blood pressure determination across various physiological conditions and may help account for individual variations in vascular properties.

The following discussion describes techniques that are implementable utilizing the systems and devices described above for multi-sensor differential measurement for blood pressure determination. Aspects of the procedure (e.g., method) may be implemented in hardware, firmware, software, or a combination thereof. The procedure is shown as a set of blocks that specify operations that may be performed by one or more devices and are not necessarily limited to the orders shown for performing the operations by the respective blocks. One or more blocks of the procedure, for instance, specify operations that are programmable by hardware (e.g., a processor, microprocessor, controller, or firmware) as executable instructions, thereby creating a special-purpose machine for carrying out an algorithm (e.g., the blood pressure estimation algorithm 118 of FIG. 1) as illustrated by the flow diagram. As a result, the instructions are storable on a computer-readable storage medium that causes the hardware to perform the algorithm. In portions of the following discussion, reference will be made to FIGS. 1-3.

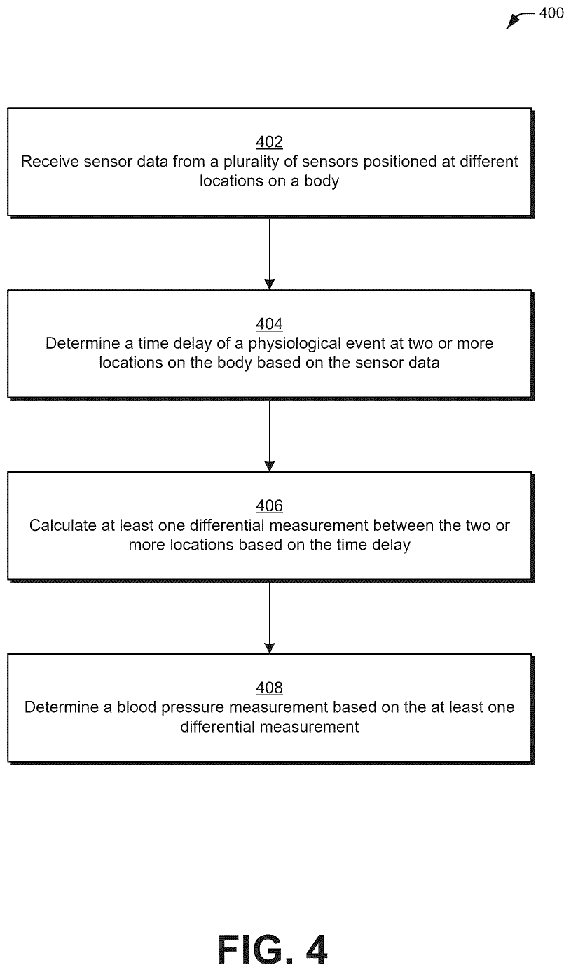

FIG. 4 illustrates a method 400 for estimating blood pressure using differential measurements. The method 400 may be implemented by the analysis platform 106 to process data from the monitoring device 104 and one or more additional monitoring devices 116, e.g., via the blood pressure estimation algorithm 118.

Sensor data is received from a plurality of sensors positioned at different locations on a body (block 402). By way of example, the analysis platform 106 may receive the sensor data as at least a portion of the measurements 108 from the monitoring device 104 and/or the additional monitoring device 116. In some implementations, two PPG sensors 310 are used, and the PPG sensors 310 are positioned at two different locations on the body. In other examples, more than two PPG sensors 310 are used. The plurality of sensors may also include the electrodes 312, impedance cardiography (ICG) sensors, accelerometers, temperature sensors, galvanic skin response sensors, blood oxygen saturation sensors, and/or pressure sensors, among others.

The sensors (e.g., the at least two PPG sensors 310), for instance, are sufficiently spaced apart from one another to capture time delay information corresponding to a propagation of a pulse wave through a vascular network of the body. It will be appreciated that the different locations on the body may include one or more of the upper chest, the lower chest, the neck (e.g., over the carotid artery), the forehead, an upper arm, a forearm, a wrist, a finger, a leg, a foot, and so forth. In one or more implementations, a first PPG sensor is positioned at a first sensor location, and a second PPG sensor is positioned at a second sensor location. The first sensor location may be a proximal location relative to the heart of the person 102, and the second sensor location may be a distal location relative to the heart. By way of example, the first PPG sensor may be positioned on the upper chest near the clavicle, and the second PPG sensor may be positioned over the carotid artery in the neck region. Alternatively, or in addition, the first PPG sensor may be positioned on the upper chest, and the second PPG sensor may be positioned on the forearm. The spatial separation between the first PPG sensor at the first location and the second PPG sensor at the second location enables the capture of time-delayed physiological signals as the pulse wave travels through the arterial system.

In some instances, at least three PPG sensors may be included. Continuing with the above example, a third PPG sensor may be positioned at a third sensor location, such as the left arm (e.g., the upper forearm or the bicep). This configuration corresponds to a distributed arrangement, such as described above with respect to the first example configuration 302 of FIG. 3, and may provide additional timing information that enables the flow of blood over the length of a vessel network to be tracked. Alternatively, or in addition, multiple PPG sensors may be arranged in a triangulation pattern (e.g., the third example configuration 306 of FIG. 3), which uses the geometric relationships between sensors to improve blood pressure determinations. The arrangement of multiple sensors in an array, for instance, may increase the number of inputs for the blood pressure estimation algorithm 118.

It will further be appreciated that multiple sensors can be placed in close proximity to each other at the same location. Clustering multiple sensors in this manner may provide redundancy in case one of the sensors does not make sufficient contact with the skin for obtaining quality PPG measurements, which can happen due to a variety of factors including sweat, hair, body motion, and so forth. Alternatively, or in addition, clustering multiple PPG sensors may provide varying emitter-detector spacing to sample blood flow at different tissue depths under the same area of skin, e.g., as described above with respect to the second example configuration 304 of FIG. 3.

In implementations where the PPG sensors 310 are arranged in a clustered or geometric pattern (e.g., as in the second example configuration 304 or the third example configuration 306 of FIG. 3), the sensor data received at block 402 may include PPG measurements obtained through cross-sensor emitter-detector pairings, where light emitted by an emitter of one PPG sensor 310 is detected by a detector of another PPG sensor 310. By way of example, light emitted by an emitter of the first PPG sensor may be detected by a detector of the second PPG sensor (and the third PPG sensor and/or a fourth PPG sensor, when included), providing PPG readings representative of different tissue depths and/or different spatial paths.

A time delay of a physiological event at two or more locations on the body is determined based on the sensor data (block 404). By way of example, the analysis platform 106 may detect characteristic features in the PPG waveforms captured by the PPG sensors 310 that correspond to the physiological event, which may be a pulse wave. A pulse wave, for instance, is a pressure wave that propagates through the arterial system following each cardiac contraction. As the left ventricle of the heart ejects blood into the aorta, the pulse wave travels through the aortic arch and branches into the various arteries of the body, including the carotid arteries, subclavian arteries, and peripheral arteries. The pulse wave causes volumetric changes in the blood vessels as it passes through each location, which are detectable by the PPG sensors 310 as changes in light reflection.

In one or more implementations, a first characteristic feature may be identified as a peak of a PPG waveform, which corresponds to a maximum blood volume at a measurement site during a cardiac cycle. Alternatively, or in addition, a second characteristic feature may be identified as a foot of the PPG waveform, which corresponds to a minimum blood volume at the measurement site and typically occurs at the onset of the pulse wave arrival. The foot of the PPG waveform may be identified as a point of maximum upslope, a minimum value, or an inflection point at the beginning of the systolic upstroke of the waveform.

In one or more implementations, the time delay of the physiological event is calculated (e.g., by the blood pressure estimation algorithm 118) for the two or more locations on the body based on respective timings of the characteristic feature in a first PPG waveform obtained at the first sensor location and in a second PPG waveform obtained at the second sensor location. By way of example, the time delay may be the PTT between the foot (or peak) of the first PPG waveform and the foot (or peak) of the second PPG waveform. Alternatively, or in addition, the time delay includes a plurality of time delays calculated relative to a common cardiac timing reference. By way of example, a first time delay may be calculated based on the characteristic feature from the first PPG waveform relative to the cardiac timing reference, and a second time delay may be calculated based on the characteristic feature from the second PPG waveform relative to the cardiac timing reference. The cardiac timing reference may be an R-peak of an ECG waveform captured by the electrodes 312, for instance, or the foot or peak of another PPG waveform. The R-peak corresponds to ventricular depolarization and provides a consistent timing marker for each cardiac cycle. When using the ECG R-peak as the cardiac timing reference, the individual time delays are sometimes referred to as pulse arrival times (PATs). Accordingly, in some instances, the first time delay may be calculated as a time interval from the R-peak of the ECG waveform to the arrival of the characteristic feature (e.g., the peak or foot of the PPG) at the first sensor location, and the second time delay may be calculated as a time interval from the R-peak of the ECG waveform to the arrival of the characteristic feature at the second sensor location.

In some cases, the sensor data may be filtered or otherwise processed to remove noise. For instance, the blood pressure estimation algorithm 118 may exclude sensor data that falls below predetermined quality thresholds or exhibits excessive noise levels. PPG signals with low amplitude or high-frequency artifacts, for example, may be filtered out to improve overall measurement accuracy. Additionally, the blood pressure estimation algorithm 118 may employ averaging techniques to enhance signal stability and reduce the impact of transient fluctuations. This approach may help mitigate the effects of respiratory variations, movement artifacts, and other sources of short-term variability in the physiological signals.

In some cases, ECG information may be used to improve accuracy by providing accurate heart rate data. By way of example, the PPG data may be segmented based on heart rate/pulse rate matching from the ECG data. The blood pressure estimation algorithm 118, for instance, may identify R-peaks in the ECG waveform to define individual cardiac cycles and segment the PPG waveforms accordingly. The blood pressure estimation algorithm 118 may verify that the pulse rate derived from the PPG waveforms matches the heart rate derived from the ECG waveform to ensure that the detected PPG features correspond to genuine pulse waves rather than motion artifacts or other noise sources. Beats for which the pulse rate does not match the heart rate may be excluded.

At least one differential measurement between the two or more locations is calculated based on the time delay (block 406). In some cases, the differential measurement refers to a differential pulse transit time (differential PTT) determined based on the PPG waveforms obtained by the two or more PPG sensors. As used herein, the differential PTT represents the time difference for the pulse wave to travel from a proximal measurement location to a distal measurement location, for example. By way of example, the blood pressure estimation algorithm 118 may calculate the differential PTT by subtracting the first time delay (e.g., measured at the first sensor location) from the second time delay (e.g., measured at the second sensor location). As a non-limiting example, the differential PTT may be calculated as a difference between pulse arrival measured at the upper chest location and the pulse arrival measured at the carotid artery. Using the differential measurement may reduce the interference of background noise that is common across the datasets of the two or more PPG sensors.

Moreover, using the differential PTT eliminates the pre-ejection period (PEP) component that is present in conventional pulse arrival time (PAT) measurements. The PEP is the time interval between ventricular depolarization (the R-peak) and the opening of the aortic valve, during which the ventricle contracts but blood has not yet been ejected into the aorta. The PEP can vary due to factors such as cardiac contractility, sympathetic activation, stress, and exercise, which introduces variability into PAT measurements that is not directly related to arterial properties or blood pressure. By calculating the differential PTT as a difference between two pulse arrival and/or transit times that both include the PEP, the PEP component cancels out, leaving a time difference attributable to the propagation of the pulse wave through different arterial paths. This differential measurement may therefore provide a more direct relationship to vascular properties and blood pressure than conventional PAT, PTT, or PEP-based measurements.

In one or more implementations, the differential PTT may be calculated using the pulse wave peak as the characteristic feature. Alternatively, the differential PTT may be calculated using the pulse wave foot as the characteristic feature. The choice between the peak and the foot may depend on signal quality, physiological conditions, and the specific activity being monitored. By way of example, during resistance band exercises, a transit time calculated using either the peak or the foot may exhibit an inverse relationship with systolic blood pressure (SBP), indicating that either feature may be suitable for blood pressure estimation. During a Valsalva maneuver, the transit time calculated using the foot may exhibit a more consistent inverse relationship with SBP compared to the peak, suggesting that the foot may be preferable for blood pressure estimation during certain physiological challenges.

In one or more implementations, multiple differential PTT values may be calculated across different sensor pairs. By way of example, if three PPG sensors are positioned at the carotid artery, the upper chest, and the arm, respectively, a first differential PTT may be calculated as a difference between a first time delay measured at the forearm and a second time delay measured at the chest, a second differential PTT may be calculated as a difference between the first time delay measured at the forearm and a third time delay measured at the carotid artery, and a third differential PTT may be calculated as a difference between the second time delay measured at the chest and the third time delay measured at the carotid artery. The blood pressure estimation algorithm 118 may select one or more of these differential PTT values based on signal quality metrics, sensor contact quality, and/or the magnitude of the differential values to optimize blood pressure estimation accuracy. The multiple differential PTT measurements may be averaged, or a median may be taken over a number of heartbeats to mitigate the effects of measurement and/or physiological artifacts.

A blood pressure measurement is determined based on the at least one differential measurement (block 408). By way of example, the blood pressure estimation algorithm 118 may apply a mapping function that relates the differential PTT to blood pressure values and/or a model that describes pulse wave velocity as a function of arterial wall properties. Pulse wave velocity, for instance, is inversely related to pulse transit time, such that shorter transit times correspond to higher pulse wave velocities. Arterial stiffness increases with increasing blood pressure, which causes the pulse wave to travel faster through the arterial system, resulting in shorter pulse transit times. Conversely, lower blood pressure is associated with more compliant (less stiff) arterial walls, which results in slower pulse wave propagation and longer pulse transit times. Therefore, the differential PTT may exhibit an inverse relationship with blood pressure, such that decreasing differential PTT values correspond to increasing blood pressure values, and increasing differential PTT values correspond to decreasing blood pressure values.

In one or more implementations, the mapping function may be a linear model, a polynomial model, an exponential model, or a machine learning model trained on reference data that includes simultaneous measurements of differential PTT and blood pressure obtained from a population of subjects. In contrast, a linear mapping function may relate blood pressure to the differential PTT through coefficients determined through calibration or training. The coefficients may be determined through regression analysis using reference blood pressure measurements obtained from oscillometric cuff devices or invasive arterial pressure monitoring during calibration procedures. The blood pressure estimation algorithm 118 may also incorporate adaptive calibration techniques to account for individual physiological differences and changes over time, which may improve the accuracy of blood pressure estimations across different users and conditions.

In one or more implementations, the blood pressure estimation algorithm 118 may implement a multi-stage processing pipeline. A first stage may include signal acquisition and preprocessing, during which raw PPG and ECG signals are filtered to remove high-frequency noise and baseline wander. A second stage may include feature extraction, during which R-peaks are identified in the ECG waveform, and pulse wave peaks or feet are identified in the PPG waveforms from each sensor location. A third stage may include quality assessment, during which signal quality metrics are calculated for each beat, and beats with insufficient quality are flagged for exclusion. A fourth stage may include the differential PTT calculation, during which time delays are calculated for each sensor location (e.g., relative to the R-peak), and differential PTT values are computed by subtracting the time delays between sensor pairs. A fifth stage may include temporal aggregation, during which differential PTT values are averaged or median-filtered over a sliding window of multiple heartbeats to reduce variability. A sixth stage may include blood pressure estimation, during which the aggregated differential PTT values are input to the mapping function to generate blood pressure estimates.

The measurements 108 generated by the method 400 may be stored in the storage device 112. The prediction system 114 may use these measurements to generate the one or more predictions 110 related to blood pressure and/or other physiological measures. In some cases, the transmitter 204 of the monitoring device 104 may transmit the blood pressure measurements to an external device for further analysis or display. The blood pressure measurements may be displayed as numerical values (e.g., systolic and/or diastolic blood pressure in mmHg), as trend graphs showing blood pressure changes over time, and/or as alerts when blood pressure values exceed and/or decrease below predetermined thresholds. The continuous or frequent non-invasive blood pressure measurements enabled by the method 400 may provide clinically valuable information about blood pressure variability during daily activities, sleep, exercise, and other physiological states that are not captured by intermittent cuff-based measurements.

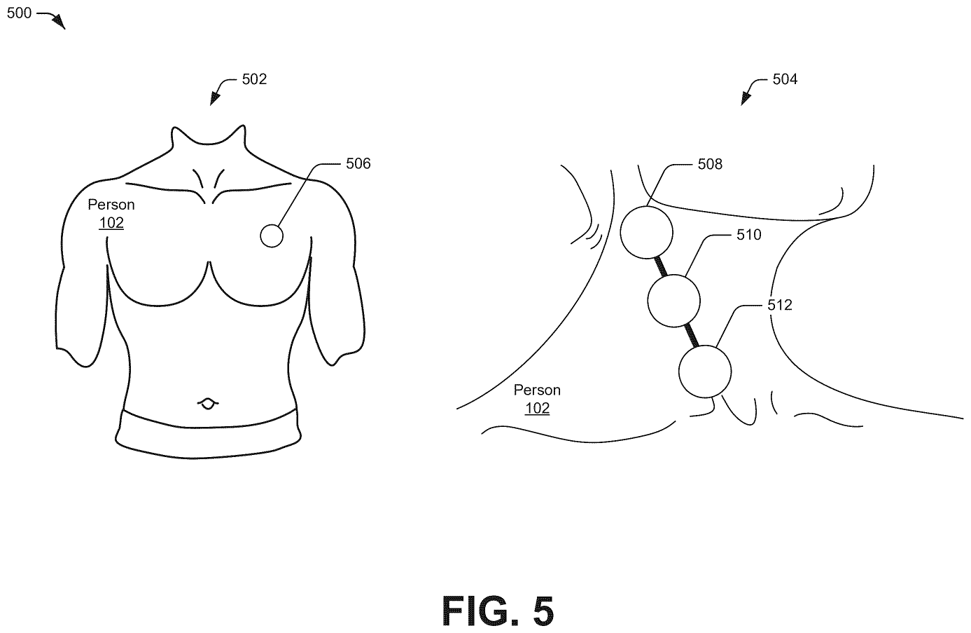

FIGS. 5-11 depict examples comparing a quality of PPG measurements and differential PTT determination using various sensor configurations in a first experimental setup.

FIG. 5 includes an example 500 of the first experimental setup for comparing PPG signal quality and PAT and/or PTT values at different sensor locations. The example 500 includes a first view 502 illustrating the torso of the person 102 and a second view 504 illustrating the neck of the person 102. The first view 502 illustrates a chest location 506 for placing the monitoring device 104 in the first experimental setup. The chest location 506 can also be described as occupying the upper left side of the chest of the person 102. As a non-limiting example, the monitoring device 104 may be placed at the chest location 506. The second view 504 illustrates a carotid top position 508 (e.g., the top position along the carotid artery at the neck), a carotid middle position 510 (e.g., the middle position along the carotid artery at the neck), and a carotid bottom position 512 (e.g., the bottom position along the carotid artery at the neck). In one or more implementations, the additional monitoring device 116 may be placed at the carotid top position 508, the carotid middle position 510, and/or the carotid bottom position 512 to create various sensor configurations for comparing the quality of PPG measurements and determining a differential PTT.

Whether included on separate monitoring devices or the same monitoring device, in the first experimental setup, a first PPG sensor is positioned at the chest location 506, and a second PPG sensor is positioned at one or more or each of the carotid top position 508, the carotid middle position 510, and the carotid bottom position 512. By way of example, the second sensor may be moved between the carotid top position 508, the carotid middle position 510, and the carotid bottom position 512 during different measurement periods for a comparative analysis of measuring the PPG signal for differential PTT determination at each of these locations. Alternatively, the PPG signal may be measured at the carotid top position 508, the carotid middle position 510, and the carotid bottom position 512 (e.g., using separate sensors, and in addition to or instead of the chest location 506) during a single measurement period.

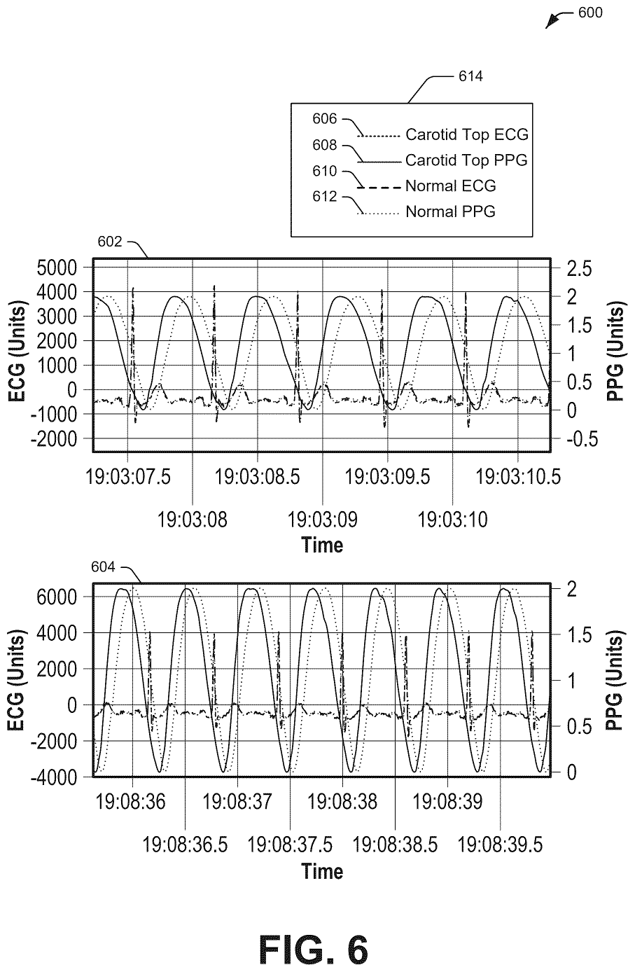

FIG. 6 depicts an example 600 that includes ECG and PPG recordings from the monitoring device 104 positioned at the chest location 506 and the additional monitoring device 116 positioned at the carotid top position 508 during a first data collection period. A first graph 602 represents data collected from the person 102 during rest, e.g., while the person 102 is in a sitting position for one minute prior to performing a movement protocol. A second graph 604 represents data collected from the person 102 at an end of the movement protocol. In one or more implementations, the movement protocol includes an ordered sequence of standing for approximately 30 seconds, lying supine for approximately 30 seconds, sitting for approximately 30 seconds, standing for approximately 30 seconds, lying supine for approximately 30 seconds, and sitting while typing for approximately two minutes, with normal neck and body movements permitted during these two minutes. It is to be appreciated that additional data may be collected during the first data collection period in addition to that shown in the first graph 602 and the second graph 604.

The first graph 602 and the second graph 604 both include a carotid top ECG plot 606, a carotid top PPG plot 608, a normal ECG plot 610, and a normal PPG plot 612, as indicated by a legend 614. The legend 614, for example, indicates the line style used to depict each plot. The carotid top ECG plot 606 is represented by a short-dashed line, the carotid top PPG plot 608 is represented by a solid line, the normal ECG plot 610 is represented by a thicker dashed line, and the normal PPG plot 612 is represented by a dotted line. The carotid top ECG plot 606 corresponds to ECG data recorded from the additional monitoring device 116 at the carotid top position 508, and the carotid top PPG plot 608 corresponds to PPG data recorded from the additional monitoring device 116 at the carotid top position 508. The normal ECG plot 610 corresponds to ECG data recorded by the monitoring device 104 at the chest location 506, and the normal PPG plot 612 corresponds to PPG data recorded by the monitoring device 104 at the chest location 506.

The first graph 602 and the second graph 604 enable a comparison of simultaneously measured signal timing and morphology across measurement locations and conditions. Beat alignment in the ECG plots indicates cardiac timing, and relative offsets between the PPG plots indicate differences in pulse arrival between the carotid top position 508 and the chest location 506. The differences in the pulse arrival time may be used to derive the differential pulse transit time. Changes in amplitude, waveform shape, and/or noise between the first graph 602 and the second graph 604 reflect posture and movement effects during the movement protocol.

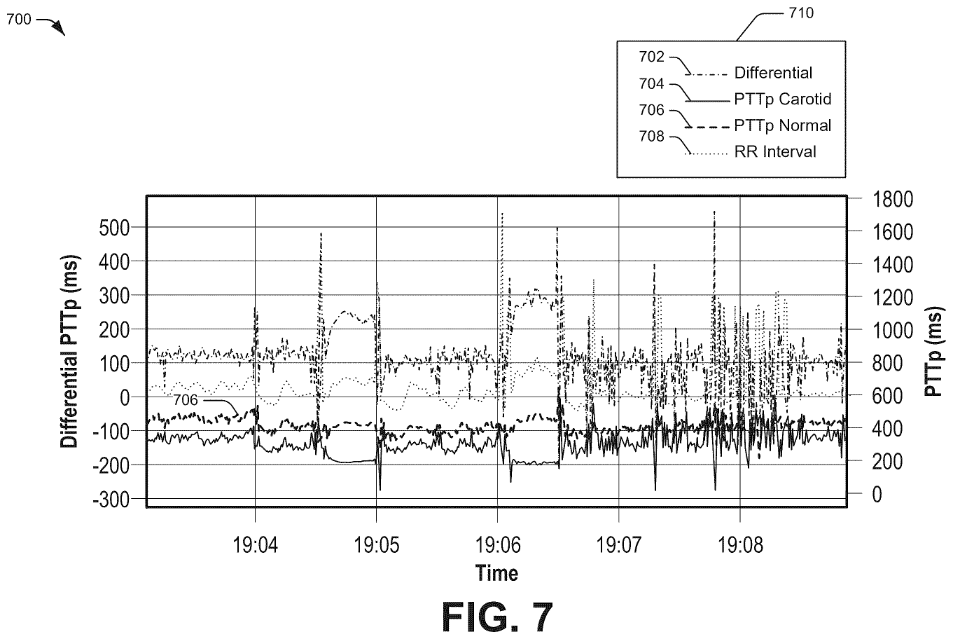

FIG. 7 depicts an example 700 that compares a pulse transit time calculated using the PPG peaks, denoted PTTp, for the first data collection period of FIG. 6. A differential plot 702 represents a differential calculated by subtracting the PTTp measured at the carotid top position 508 by the additional monitoring device 116 from the PTTp measured at the chest location 506 by the monitoring device 104. A PTTp carotid plot 704 depicts the PTTp measured by the additional monitoring device 116 at the carotid top position 508. A PTTp normal plot 706 depicts the PTTp measured by the monitoring device 104 at the chest location 506. An RR interval plot 708 depicts an interval between adjacent R-waves of the ECG data. A legend 710 identifies the line styles, with the differential plot 702 shown as a dash-dot line, the PTTp carotid plot 704 as a solid line, the PTTp normal plot 706 as a thicker dashed line, and the RR interval plot 708 as a dotted line.

The example 700 shows temporal relationships between differential timing measurements and heart rate variability, with periods of increased differential values corresponding to changes in the RR interval plot 708, indicating how pulse transit timing varies with cardiac cycle length during the movement protocol. In this example, the mean differential PTTp for the carotid top position 508 was calculated as 112.4 milliseconds (ms).

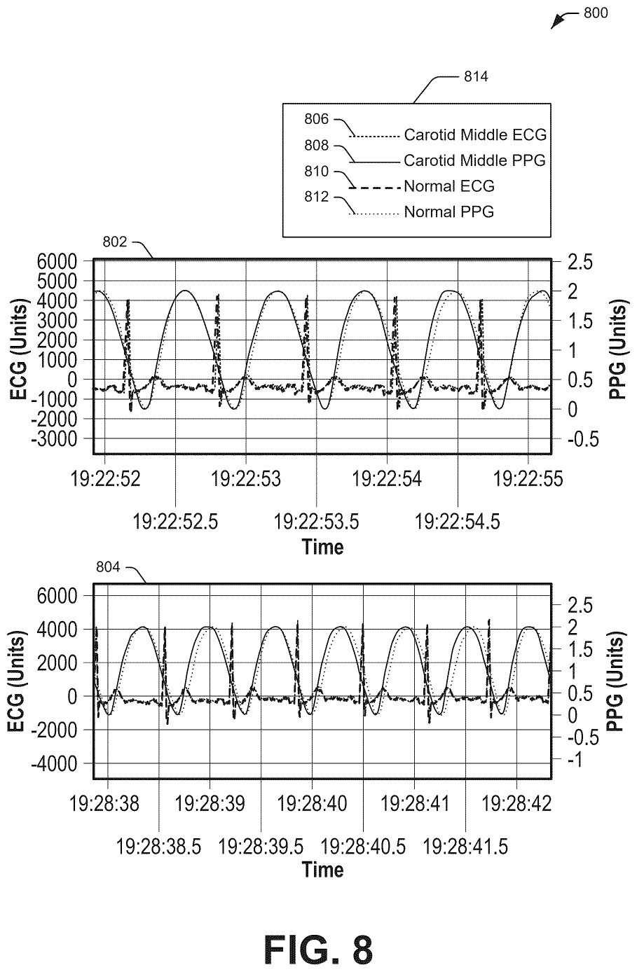

FIG. 8 depicts an example 800 that includes ECG and PPG recordings from the monitoring device 104 positioned at the chest location 506 and the additional monitoring device 116 positioned at the carotid middle position 510 during a second data collection period. A first graph 802 represents data collected from the person 102 during rest prior to performing the movement protocol described with respect to FIG. 6. A second graph 804 represents data collected from the person 102 at the end of the movement protocol. It is to be appreciated that additional data may be collected during the second data collection period in addition to that shown in the first graph 802 and the second graph 804.

The first graph 802 and the second graph 804 both include a carotid middle ECG plot 806, a carotid middle PPG plot 808, a normal ECG plot 810, and a normal PPG plot 812, as indicated by a legend 814. The legend 814 indicates the line style used to depict each plot. The carotid middle ECG plot 806 corresponds to ECG data recorded by the additional monitoring device 116 at the carotid middle position 510, and the carotid middle PPG plot 808 corresponds to PPG data recorded by the additional monitoring device 116 at the carotid middle position 510. The normal ECG plot 810 corresponds to ECG data recorded by the monitoring device 104 at the chest location 506, and the normal PPG plot 812 corresponds to PPG data recorded by the monitoring device 104 at the chest location 506.

Similar to the first graph 602 and the second graph 604 of FIG. 6, the first graph 802 and the second graph 804 enable a comparison of simultaneously measured signal timing and morphology across measurement locations and conditions to derive the differential pulse transit time for the carotid middle position 510.

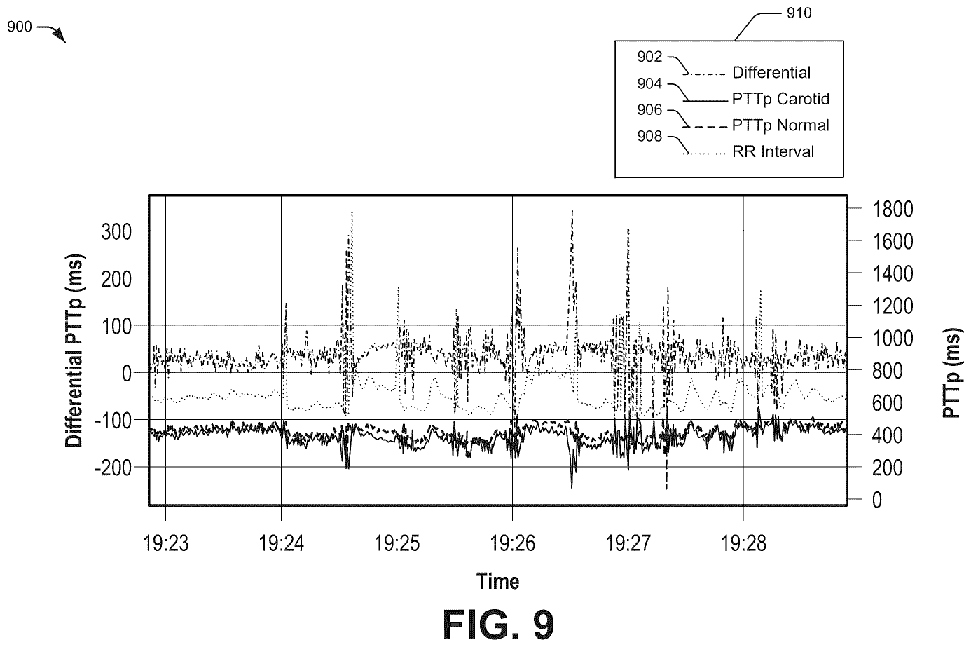

FIG. 9 depicts an example 900 that compares pulse transit time calculated using PPG peaks, denoted PTTp, for the second data collection period of FIG. 8. A differential plot 902 represents a differential calculated by subtracting the PTTp measured at the carotid middle position 510 by the additional monitoring device 116 from the PTTp measured at the chest location 506 by the monitoring device 104. A PTTp carotid plot 904 depicts the PTTp measured by the additional monitoring device 116 at the carotid middle position 510. A PTTp normal plot 906 depicts the PTTp measured by the monitoring device 104 at the chest location 506. An RR interval plot 908 depicts an interval between adjacent R-waves of the ECG data. A legend 910 identifies the line styles, such as described above with respect to FIG. 7.

The example 900 shows temporal relationships between differential timing measurements and heart rate variability for the carotid middle position 510. In this example, the mean differential PTTp for the carotid middle position 510 was calculated as 32.1 ms.

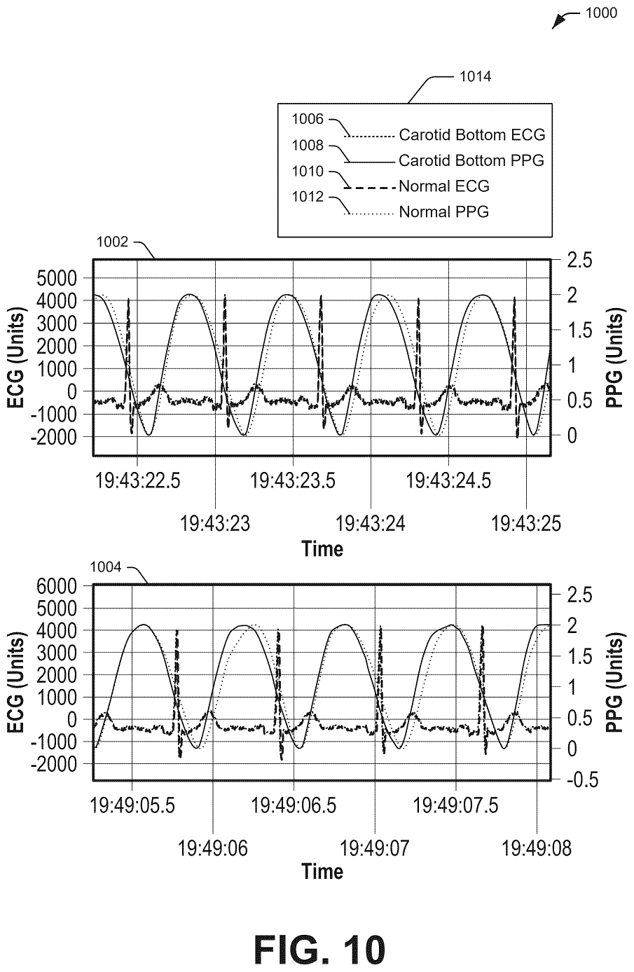

FIG. 10 depicts an example 1000 that includes ECG and PPG recordings from the monitoring device 104 positioned at the chest location 506 and the additional monitoring device 116 positioned at the carotid bottom position 512 during a third data collection period. A first graph 1002 represents data collected from the person 102 during rest prior to performing the movement protocol described with respect to FIG. 6. A second graph 1004 represents data collected from the person 102 at the end of the movement protocol. It is to be appreciated that additional data may be collected during the third data collection period in addition to that shown in the first graph 1002 and the second graph 1004.

The first graph 1002 and the second graph 1004 both include a carotid bottom ECG plot 1006, a carotid bottom PPG plot 1008, a normal ECG plot 1010, and a normal PPG plot 1012, as indicated by a legend 1014. The legend 1014 indicates the line style used to depict each plot, such as described above with respect to FIG. 6. The carotid bottom ECG plot 1006 corresponds to ECG data recorded by the additional monitoring device 116 at the carotid bottom position 512, and the carotid bottom PPG plot 1008 corresponds to PPG data recorded by the additional monitoring device 116 at the carotid bottom position 512. The normal ECG plot 1010 corresponds to ECG data recorded by the monitoring device 104 at the chest location 506, and the normal PPG plot 1012 corresponds to PPG data recorded by the monitoring device 104 at the chest location 506.

Similar to the first graph 602 and the second graph 604 of FIG. 6 and the first graph 802 and the second graph 804 of FIG. 8, the first graph 1002 and the second graph 1004 enable a comparison of simultaneously measured signal timing and morphology across measurement locations and conditions to derive the differential pulse transit time for the carotid bottom position 512.

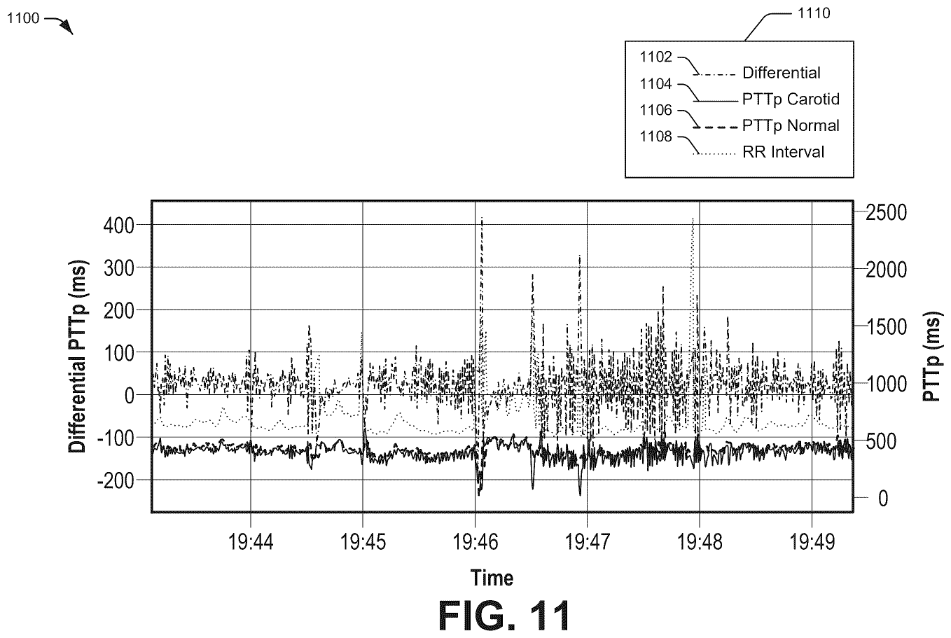

FIG. 11 depicts an example 1100 that compares pulse transit time calculated by using PPG peaks, denoted PTTp, for the third data collection period of FIG. 10. A differential plot 1102 represents a differential calculated by subtracting the PTTp measured at the carotid bottom position 512 by the additional monitoring device 116 from the PTTp measured at the chest location 506 by the monitoring device 104. A PTTp carotid plot 1104 depicts the PTTp measured by the additional monitoring device 116 at the carotid bottom position 512. A PTTp normal plot 1106 depicts the PTTp measured by the monitoring device 104 at the chest location 506. An RR interval plot 1108 depicts an interval between adjacent R-waves of the ECG data. A legend 1110 identifies the line styles, such as described above with respect to FIG. 7.

The example 1100 shows temporal relationships between differential timing measurements and heart rate variability for the carotid bottom position 512. In this example, the mean differential PTTp for the carotid bottom position 512 was calculated as 22.2 ms.

In comparing FIGS. 6-11, for all three carotid locations the average differential PTTp values were positive. This suggests that a majority of carotid PPG peaks arrived prior to corresponding chest PPG peaks. Although the carotid artery may appear farther from the heart visually, a pulse wave path along the carotid artery may be more streamlined. The pulse wave travels from the aortic arch to the common carotid artery, and the common carotid artery moves “radially” outward toward the skin surface as it progresses to the brain. A distance between the artery and capillaries at the neck may be relatively short and less resistive compared to the chest location. To arrive at the chest PPG location, the pulse wave travels from the heart to the aortic arch, curls around the aortic arch, and branches into arterioles and capillaries. This curling travel along the aortic arch may not be captured by a simple visual distance. A higher resistance of this path may correspond to the longer transit times observed in the example 700 of FIG. 7, the example 900 of FIG. 9, and the example 1100 of FIG. 11.

PTTp differences at the carotid middle position 510 and the carotid bottom position 512 were smaller than the differences at the carotid top position 508. The mean difference between the PTTp differences at the carotid top position 508 versus the carotid middle position 510 was 80.3 ms, and the mean difference between PTTp differences at the carotid top position 508 vs the carotid bottom position 512 was 90.2 ms. The mean difference between PTTp differences at the carotid middle position 510 versus the carotid bottom position 512 was small at 9.9 ms. The differences at the carotid top position 508 may not be proportional to the distance between the locations. This may be due to the close proximity of the artery to the skin at the top location. At the carotid top position 508, the carotid top PPG plot 608 showed better quality compared to other locations. By way of example, dicrotic notches were clear during rest, and there was very little high frequency noise, as illustrated in the first graph 602 of FIG. 6. The differences in morphology between the PPG signal at different locations suggest that carotid PPG peaks are being identified earlier than they otherwise would have been with a PPG signal that exhibited a more rounded out (e.g., more low frequency) behavior.

Given motion artifact susceptibility at distal locations such as the wrist, evaluation of a more distant neck location from the chest location 506 was beneficial. Of the three carotid sites, the carotid top position 508 exhibited the largest mean differential PTTp across the movement protocol described with respect to FIG. 6, as shown in the differential plot 702 of FIG. 7.

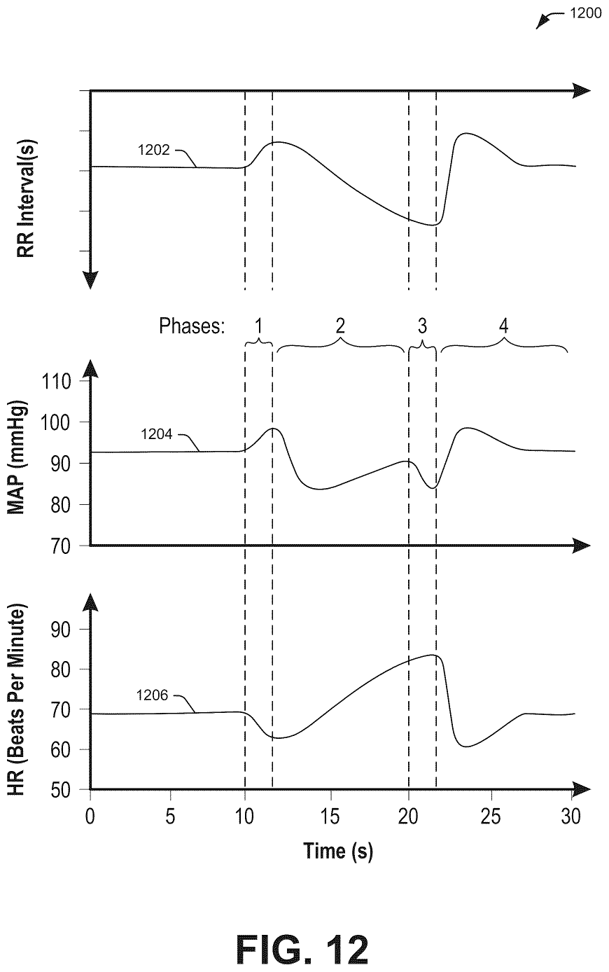

FIGS. 12-14 depict examples comparing a quality of PPG measurements and differential PTT determination in a second experimental setup.

FIG. 12 illustrates an example 1200 of expected measurement behaviors during a Valsalva maneuver. A Valsalva maneuver is a breathing action in which a person forcibly exhales against a closed airway, typically by attempting to exhale while keeping the mouth and nose closed or by exhaling against resistance. The maneuver induces characteristic changes in intrathoracic pressure, venous return, cardiac output, and blood pressure that progress through four distinct phases. Phase 1 begins when positive airway pressure is applied, causing an immediate increase in intrathoracic pressure that transiently elevates blood pressure. Phase 2 occurs during sustained strain, where reduced venous return decreases cardiac output and blood pressure gradually falls, triggering compensatory increases in heart rate and peripheral resistance. Phase 3 begins when the positive airway pressure is released, resulting in a brief drop in blood pressure as intrathoracic pressure normalizes and blood pools in the pulmonary circulation. Phase 4 follows as venous return and cardiac output recover, leading to an overshoot in blood pressure above baseline levels and a reflex bradycardia as baroreceptors respond to the elevated pressure. The Valsalva maneuver provides a controlled physiological challenge that allows evaluation of cardiovascular reflexes and hemodynamic responses.

The example 1200 includes an RR interval plot 1202, a mean arterial pressure (MAP) plot 1204, and a heart rate (HR) plot 1206 in a time-aligned manner. The four phases described above are labeled as Phase 1 through Phase 4, as separated by vertical dashed lines located at about 10 seconds, about 12 seconds, about 20 seconds, and about 22 seconds. The RR interval plot 1202 shows a stable baseline through about 10 seconds, an increase during Phase 1, a decrease during Phase 2, a return toward the baseline during Phase 3, and an increase during Phase 4 before returning to the baseline value. The MAP plot 1204 shows a baseline prior to Phase 1, an increase at the start of Phase 1, a gradual decrease and recovery during Phase 2, a dip during Phase 3, and a peak during Phase 4 before returning to the baseline. The HR plot 1206 shows a baseline before Phase 1, a decrease during Phase 1, an increase during Phase 2, a peak during Phase 3, and a decrease during Phase 4 before a return to the baseline. Accordingly, the RR interval plot 1202, the MAP plot 1204, and the HR plot 1206 respond to the physiological changes described above during the Valsalva maneuver.

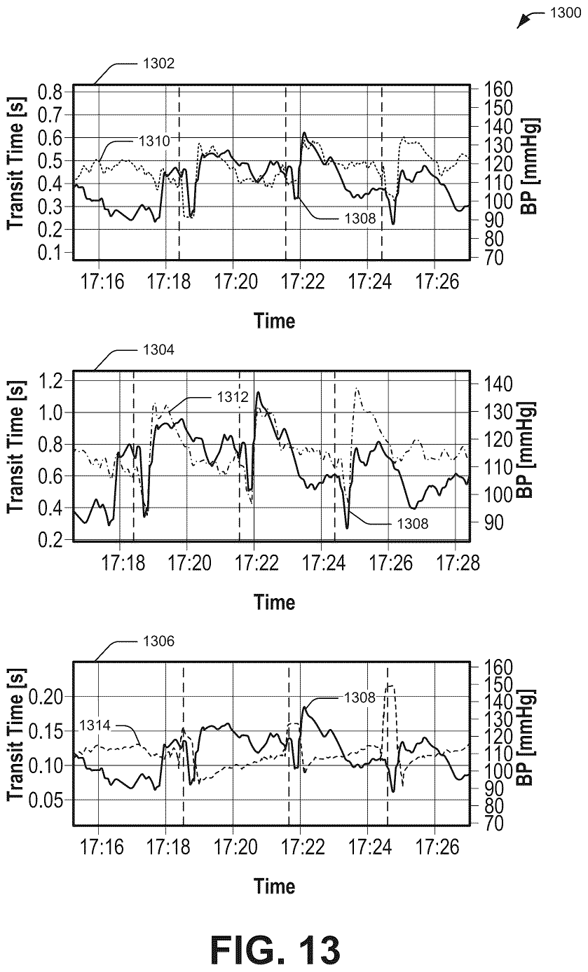

FIG. 13 illustrates examples 1300 comparing pulse transit time metrics and blood pressure data during a Valsalva maneuver. The examples 1300 include a first graph 1302, a second graph 1304, and a third graph 1306 displaying synchronized measurements. Dashed lines represent the start of each Valsalva maneuver. The first graph 1302 includes a systolic blood pressure (SBP) plot 1308 and a PTTp plot 1310. The second graph 1304 includes the SBP plot 1308 and a PTTf plot 1312, where “PTTf” refers to a pulse transit time calculated using the foot of PPG pulse waveforms. The third graph 1306 includes the SBP plot 1308 and an RR interval plot 1314. The PTTp plot 1310, the PTTf plot 1312, and the RR interval plot 1314 are shown against a vertical axis labeled Transit Time (in seconds, s), and the SBP plot 1308 is shown against a vertical axis labeled blood pressure (BP) in mmHg.

In some implementations associated with the examples 1300, several differential pulse transit time metrics may be calculated and compared (e.g., by the blood pressure estimation algorithm 118). A first differential PTT may be calculated as a forearm-measured PTT (peak or foot) minus a chest-measured PTT (peak or foot). A second differential PTT may be calculated as the forearm-measured PTT (peak or foot) minus an aortic valve-referenced PTT (peak or foot). A third differential PTT may be calculated as the aortic valve-referenced PTT (peak or foot) minus the chest-measured PTT (peak or foot).

The examples 1300 show relationships between vascular transit measures and pressure changes across the phases of the Valsalva maneuver. While the PTTp plot 1310 and the RR interval plot 1314 were expected to be inversely correlated with the SBP plot 1308, the PTTp plot 1310 and the RR interval plot 1314 generally increase and decrease according to the SBP plot 1308. Accordingly, the PTTf may be selected to calculate the differential PTT in this example.

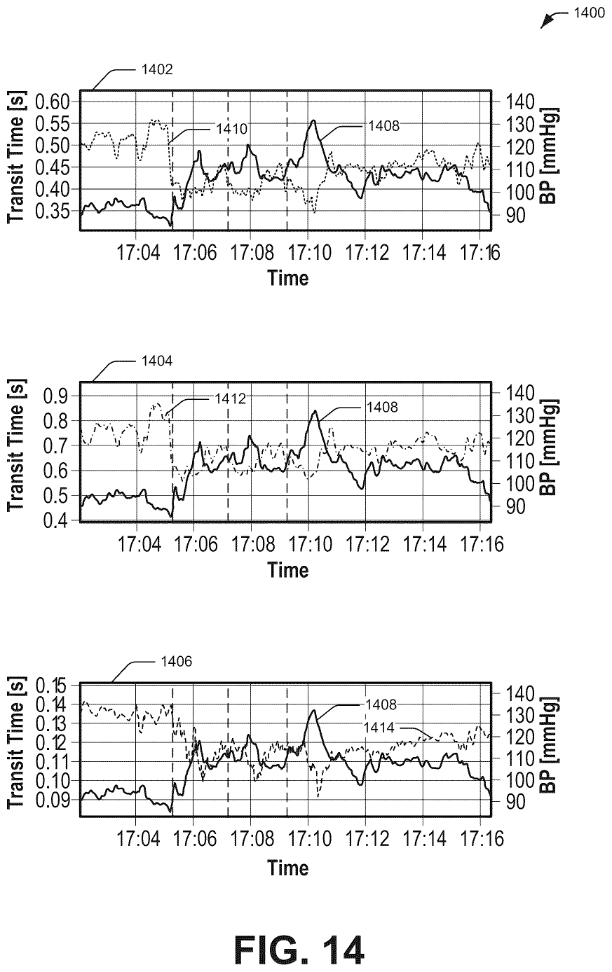

FIG. 14 depicts examples 1400 comparing pulse transit time metrics and blood pressure data during resistance band exercises. The examples 1400 include a first graph 1402, a second graph 1404, and a third graph 1406 displaying synchronized measurements. Dashed lines represent the start of each resistance band exercise. The first graph 1402 includes a systolic blood pressure (SBP) plot 1408 and a PTTp plot 1410. The second graph 1404 includes the SBP plot 1408 and a PTTf plot 1412. The third graph 1406 includes the SBP plot 1408 and an RR interval plot 1414. The PTTp plot 1410, the PTTf plot 1412, and the RR interval plot 1414 are shown against a vertical axis labeled Transit Time (in seconds, s), and the SBP plot 1408 is shown against a vertical axis labeled blood pressure (BP) in mmHg.

The examples 1400 show temporal relationships between differential timing measurements and blood pressure, with the PTTp plot 1410, the PTTf plot 1412, and the RR interval plot 1414 inversely related to the SBP plot 1408 during the resistance band exercises. Accordingly, the PTTp and/or the PTTf may be used to determine the differential PTT in this example.

### Machine Learning and AI in Multi-Sensor Differential Measurement for Blood Pressure Determination

The previous examples describe various instances of artificial intelligence (“AI”) models or machine learning models, such as with respect to the blood pressure estimation algorithm 118 and the prediction system 114. In one or more examples, an AI model, e.g., a machine learning model, refers to a computer representation that is tunable (e.g., through training and retraining) based on inputs to approximate unknown functions, automatically and without user intervention, without being actively programmed by a user. For instance, the term machine learning model includes a model that utilizes algorithms to learn from, and make predictions on, known data by analyzing training data to learn and relearn to generate outputs that reflect patterns and attributes of the training data.

In the context of multi-sensor differential blood pressure estimation, machine learning models are implementable (e.g., by one or more processing devices of the analysis platform 106) to analyze physiological data patterns, such as to identify pulse features, determine signal quality and sensor pairing, and/or generate blood pressure estimates. For example, the blood pressure estimation algorithm 118 and/or the prediction system 114 may utilize one or more machine learning models to process physiological data such as PPG signals, ECG-derived timing for segmentation, accelerometer signals, heart rate variability, respiratory patterns, and/or other measurements collected by the monitoring device 104 and/or the additional monitoring device 116. Examples of machine learning models applicable to timing-feature extraction, sensor-pair selection, and blood pressure estimation include neural networks, convolutional neural networks (CNNs) such as for analyzing waveform data and identifying pulse features, long short-term memory (LSTM) neural networks such as to analyze temporal physiological patterns and beat-to-beat dynamics, generative adversarial networks (GANs), decision trees (e.g., for classification of signal-quality states), support vector machines, linear regression, logistic regression for quality gating, Bayesian networks, random forest learning for feature importance, dimensionality reduction algorithms, boosting algorithms, deep learning neural networks, and so forth.

A machine learning model, for instance, is configurable using a plurality of layers having, respectively, a plurality of nodes. The plurality of layers is configurable to include an input layer, an output layer, and one or more hidden layers. In the context of differential blood pressure estimation, the input layer may receive various physiological parameters from the measurements 108, such as per-beat PPG features from multiple sites, PTTp and PTTf at each site, differential PTT features between sites, RR intervals, accelerometer data, heart rate patterns, respiratory signals, motion data, and/or timing information synchronized across devices. The hidden layers, for instance, process these inputs through weighted connections to identify complex patterns indicative of vascular state and pressure, e.g., patterns that are not detectable using conventional threshold-based methods. The output layer may produce blood pressure estimates, quality or activation/deactivation signals for selective computation, confidence scores, and/or generate the one or more predictions 110 that incorporate selectively processed physiological data from multiple sensors. Calculations are performed by the nodes within the layers via hidden states through a system of weighted connections that are “learned” during training of the machine learning model to implement a variety of timing-feature extraction, quality assessment, and blood pressure estimation tasks.

In order to train the machine learning model for differential blood pressure estimation, training data are received that provide examples of “what is to be learned” by the machine learning model, i.e., as a basis to learn patterns from the data. For blood pressure estimation applications, the training data may include labeled datasets of physiological measurements with reference blood pressure values, such as cuff-based measurements and, when available, invasive arterial pressure measurements, along with synchronized PPG, optional ECG, accelerometer data, and derived timing features (e.g., PTTp, PTTf, differential PTT). A machine learning system that includes the machine learning model, for instance, collects and preprocesses the training data that include input features (e.g., multi-site PPG waveforms, PTTp/PTTf per site, differential PTTs, RR intervals, accelerometer signals) and corresponding target labels (e.g., systolic, diastolic, and/or mean arterial pressure).

The machine learning system is further operable to initialize various parameters of the machine learning model, which are usable by the machine learning model as internal variables to represent and process information during training. These parameters are further usable to represent inferences gained through training. In one or more implementations, the training data are separated into batches to improve processing and optimization efficiency of the parameters of the machine learning model during training, which may be beneficial for model accuracy when processing large volumes of physiological time-series data from multiple devices with varying sampling rates and temporal characteristics.

The training data are then received by the machine learning model as inputs and used to generate predictions based on a current state of parameters of layers and corresponding nodes of the model, a result of which is output as output data, e.g., a blood pressure estimate, a signal-quality classification, a sensor-pair selection probability, or the like. For example, the analysis platform 106 includes a machine learning model that is trained to recognize patterns in differential timing features and waveform morphology that correlate with blood pressure, which enables the blood pressure estimation algorithm 118 and the prediction system 114 to generate accurate estimates while selectively utilizing higher-quality sensor pairs and timing segments.

Training of the machine learning model can include calculation of a loss function to quantify a loss associated with operations performed by nodes of the machine learning model. The loss function is configurable in various ways to control operation or functionality of the machine learning model. For instance, the loss function may be designed to prioritize accuracy in blood pressure estimation while minimizing unwarranted errors. Calculation of the loss function, for instance, includes comparing a difference between predictions specified in the output data (e.g., predicted systolic/diastolic/mean arterial pressure and/or signal quality decisions) with target labels specified by the training data (e.g., reference blood pressure or verified quality states). The loss function is configurable in a variety of ways, examples of which include least-squares or Huber losses for continuous pressure estimation, cross-entropy loss for quality classification tasks, custom loss functions that incorporate power efficiency requirements or data collection priorities for wearable operation, temporal-consistency constraints, and so forth.

The training data are usable to support a variety of usage scenarios in differential blood pressure estimation. For example, the machine learning model can be trained to detect specific patterns in multi-site PPG that enable robust PTTp/PTTf extraction, identify motion patterns that inform quality gating, recognize posture or maneuver-related changes (e.g., during daily activity or physiologic challenges), and/or detect subtle timing changes that may improve estimation precision. The models can be configured to operate within the computational constraints of real-time inference while providing accurate estimates. The models can further be reconfigured, e.g., with expanded capabilities, for more sophisticated analysis when processing historical data or performing calibration. This adaptive approach enables efficient use of computational resources devoted to machine learning processes while ensuring comprehensive differential timing analysis capabilities are available when needed for accurate multi-sensor blood pressure estimation across multiple devices and measurement modalities.

It should be understood that many variations are possible based on the disclosure herein. Although features and elements are described above in particular combinations, each feature or element is usable alone without the other features and elements or in various combinations with or without other features and elements.

What is claimed is:

1. A method for estimating blood pressure, comprising:
receiving sensor data from a plurality of sensors positioned at different locations on a body;
determining a time delay of a physiological event at two or more locations on the body based on the sensor data;
calculating at least one differential measurement between the two or more locations based on the time delay; and
determining a blood pressure measurement based on the at least one differential measurement.

2. The method of claim 1, wherein the plurality of sensors comprises at least two photoplethysmography (PPG) sensors positioned at the different locations on the body.

3. The method of claim 2, wherein the plurality of sensors further comprises at least one of an electrocardiography (ECG) sensor, an impedance cardiography (ICG) sensor, or an accelerometry sensor.

4. The method of claim 2, wherein the at least two PPG sensors include a first PPG sensor at a proximal position relative to a heart and a second PPG sensor at a distal position relative to the heart.

5. The method of claim 1, wherein determining the time delay of the physiological event at the two or more locations on the body based on the sensor data comprises:
detecting a characteristic feature in first sensor data obtained at a first of the two or more locations on the body;
detecting the characteristic feature in second sensor data obtained at a second of the two or more locations on the body; and
determining the time delay of the physiological event based on respective timings of the detected characteristic feature in the first sensor data and the second sensor data.

6. The method of claim 1, wherein the physiological event comprises an arrival of a pulse wave at a corresponding location of the two or more locations on the body.

7. The method of claim 1, wherein the different locations on the body include one or more of an upper chest, a lower chest, a neck region, a forehead, an upper arm, a forearm, a wrist, a finger, a leg, or a foot.

8. The method of claim 1, wherein the plurality of sensors includes at least three PPG sensors.

9. The method of claim 8, wherein the at least three PPG sensors are positioned in a configuration to triangulate the blood pressure.

10. The method of claim 1, wherein the plurality of sensors includes at least two PPG sensors positioned to obtain light reflectance measurements received from different depths of a same area on the body.

11. The method of claim 1, further comprising processing the sensor data prior to determining the time delay by:
filtering the sensor data to remove noise; and
removing portions of the sensor data that are below a quality threshold.

12. The method of claim 1, wherein determining the blood pressure measurement based on the at least one differential measurement comprises applying one or more statistical analyses to the at least one differential measurement over a plurality of heartbeats.

13. A system for estimating blood pressure, comprising:
a plurality of sensors configured for placement at different locations on a body, including:
a first photoplethysmography (PPG) sensor configured to be positioned at a first location on the body; and
a second PPG sensor configured to be positioned at a second location on the body; and
at least one processor configured to:
determine a differential measurement based on a time delay of a physiological event that is determined based on a characteristic feature detected by the first PPG sensor and a corresponding characteristic feature detected by the second PPG sensor; and
determine a blood pressure measurement based on the differential measurement.

14. The system of claim 13, wherein the plurality of sensors further comprises at least one electrocardiography (ECG) sensor.

15. The system of claim 13, wherein the plurality of sensors further includes a third PPG sensor configured to be positioned at a third location on the body that is selected relative to the first location and the second location to enable triangulation of the blood pressure measurement.

16. The system of claim 13, wherein the different locations include one or more of an upper chest, a lower chest, a neck region, a forehead, an upper arm, a forearm, a wrist, a finger, a leg, or a foot.

17. The system of claim 13, wherein to determine the differential measurement based on the time delay of the physiological event, the at least one processor is further configured to:
detect the characteristic feature in a first PPG waveform obtained by the first PPG sensor;
detect the corresponding characteristic feature in a second PPG waveform obtained by the second PPG sensor; and
calculate the differential measurement based on respective timings of the characteristic feature in the first PPG waveform and the corresponding characteristic feature in the second PPG waveform.

18. A method for estimating blood pressure, comprising:
receiving a first photoplethysmography (PPG) waveform from a first PPG sensor and a second PPG waveform from a second PPG sensor that is spaced apart from the first PPG sensor on a body;
determining a time delay of a physiological event based on respective timings of a characteristic feature in the first PPG waveform and a corresponding characteristic feature in the second PPG waveform;
calculating a differential measurement based on the time delay; and
determining a blood pressure measurement based on the differential measurement.

19. The method of claim 18, wherein determining the time delay of the physiological event based on the respective timings of the characteristic feature in the first PPG waveform and the corresponding characteristic feature in the second PPG waveform comprises:
determining, based on the first PPG waveform, a first pulse transit time (PTT) corresponding to a first time delay for a pulse wave to reach the first PPG sensor; and
determining, based on the second PPG waveform, a second PTT corresponding to a second time delay for the pulse wave to reach the second PPG sensor, and wherein the differential measurement is a difference between the first PTT and the second PTT.

20. The method of claim 18, wherein determining the time delay of the physiological event based on the respective timings of the characteristic feature in the first PPG waveform and the corresponding characteristic feature in the second PPG waveform comprises calculating a time difference between the characteristic feature in the first PPG waveform and the corresponding characteristic feature in the second PPG waveform.
